(19) United States
(12) Patent Application Publication
(10) Pub. No.: US 2025/0022610 A1
(43) Pub. Date: Jan. 16, 2025

(54) METHOD FOR ASSESSING AEROSOL INFECTION RISK, PROGRAM FOR ASSESSING AEROSOL INFECTION RISK, DEVICE FOR ASSESSING AEROSOL INFECTION RISK, AIR CLEANING SYSTEM, METHOD FOR ASSESSING HAZARDOUS DUST RISK, PROGRAM FOR ASSESSING HAZARDOUS DUST RISK, AND DEVICE FOR ASSESSING HAZARDOUS DUST RISK

(71) Applicant: NATIONAL SECURITY SERVICE LLC, Tokyo (JP)

(72) Inventor: Tetsuya MIZUNO, Tokyo (JP)

(73) Assignee: NATIONAL SECURITY SERVICE LLC, Tokyo (JP)

(21) Appl. No.: 18/710,607

(22) PCT Filed: Nov. 15, 2022
(86) PCT No.: PCT/JP2022/042375

§ 371 Date: May 15, 2024

(30) Foreign Application Priority Data
Nov. 17, 2021 (JP) 2021-187289
May 11, 2022 (JP) 2022-077949
Sep. 6, 2022 (JP) 2022-141785

(51) Int. Cl. G16H 50/50; G16H 15/00

(52) U.S. Cl. CPC G16H 50/50; G16H 15/00

(57) ABSTRACT

A method for evaluating an aerosol infection risk includes; a release step releasing fine particles having an average particle size of 10 μm or less into a space having a predetermined volume so that the amount in the space is at least 50 mg/m3; an identifying step includes measuring the number of fine particles in the space after the releasing step, calculating a specific elapsed time until the number of fine particles decreases to a predetermined number based on a relationship between the elapsed time from the end of the releasing step and the number of fine particles measured at each elapsed time, and identifying a reduction capacity to reduce fine particles in the space from the specific elapsed time; and, evaluation steps of evaluating that there is an aerosol infection risk in the space when the reduction capacity is less than the standard reduction capacity required for the space.

### CROSS-REFERENCE TO THE RELATED APPLICATIONS

This application claims priority to (1) Japanese Patent Application No. 2021-187289 filed on Nov. 17, 2021, (2) Japanese Patent Application No. 2022-77949 filed on May 11, 2022, and (3) Japanese Patent Application No. 2022-141785 filed on Sep. 6, 2022. The entire disclosure of Japanese Patent Applications above is hereby incorporated herein by reference.

The present inventions rerate to a method for evaluating an aerosol infection risk, an evaluating program for evaluating an aerosol infection risk, an evaluating apparatus for evaluating an aerosol infection risk, an air purification system, a method for evaluating a dust damage risk, an evaluating program for evaluating a dust damage risk, and an evaluating apparatus for evaluating a dust damage risk.

### BACKGROUND

Since 2019, the new coronavirus infection (COVID-19) has been raging around the world. Airborne infection is one of the main causes of the new coronavirus infection. Therefore, by avoiding the so-called three Cs (closed spaces, crowds, and close contact), the risks of airborne infections and cluster outbreaks can be reduced. In other words, avoiding the Three Cs is an effective measure to prevent the spread of the new coronavirus.

The government, related ministries, local governments, etc. recommend that people refrain from going to and from facilities where people gather, such as restaurants, commercial facilities, and entertainment facilities, in order to avoid the Three Cs. In particular, restaurants are small spaces where people are likely to eat, drink, and talk without masks for long periods of time, increasing the risk of airborne infection. As a result, restaurants are being forced to shorten their business hours or close, the number of customers has plummeted, and they are being forced to face tough business conditions. However, if restaurants can take sufficient measures against the Three Cs and objectively prove that the restaurant can be used safely and securely, it is possible to improve this situation (shortened business hours and closures). This can be one method.

One of the three C's countermeasures is to ensure adequate ventilation within the store and remove viruses from within the store. Conventionally, the ability of a particular store to remove viruses from inside the store has been determined based on the store's ventilation capacity.

For example, the following four methods are known as methods for measuring the ventilation capacity of closed spaces such as stores.
(1) A method of installing an anemometer, etc. at the exhaust port of a ventilation system leading to the space to be measured, measuring the air volume at the exhaust port, and calculating the ventilation volume of the space.
(2) A method in which the space to be measured is ventilated, the concentration of CO2 gas emitted by humans is detected with a sensor, etc., and the ventilation amount of the space is calculated.
(3) After filling the space to be measured with tracer gas, ventilate the space, adsorb the tracer gas on an adsorbent installed in the space, quantitatively analyze the quantity of the adsorbed tracer gas, A method of calculating the ventilation amount of the space from the quantity of tracer gas (see Patent Documents 1 and 2)
(4) A method in which the space to be measured is filled with mist particles, the space is ventilated, and the ventilation amount of the space is calculated from image data taken by a photographing means installed in the space (Patent Document 3) reference)

### PRIOR ART DOCUMENTS

### Patent document

[Patent Document 1] Japanese Patent Application Publication No. 2003-42491
[Patent Document 2] Japanese Patent Application Publication No. 2007-10363
[Patent Document 3] Japanese Patent Application Publication No. 2006-250779

### SUMMARY OF THE INVENTION

### Problem to be Solved by the Invention

By the way, ventilation is a method of exhausting the virus-contaminated air inside the store outside the store and bringing in fresh air, and is just one way to reduce the risk of infection. For example, methods such as circulating the air inside the store with a circulator or air conditioner or using an air purifier can also reduce the concentration of the virus and suppress the risk of infection in the store. However, these methods cannot properly evaluate the effects of air circulation and air purifiers.

Specifically, (1) the method using anemometers and funnels is suitable for evaluating the capacity of the store ventilation system itself, but it has the following problems.
(1A) It is not possible to evaluate ventilation ability by opening windows or purification ability by circulators, air conditioners, air purifiers, etc.
(1B) The hassle of installing wind meters and funnels at each exhaust port is cumbersome.
(1C) Even if the capacity of the ventilation system is evaluated, it is not possible to evaluate the decrease in ventilation efficiency due to so-called “short circuit”.

Here, a “short circuit” refers to the fact that the intake and exhaust ports are located close to each other, causing fresh air taken in to the room to be exhausted without reaching the room, or conversely to cause contaminated air to be exhausted when it should have been exhausted. This is a phenomenon in which ventilation efficiency decreases due to air being taken in again from the air intake. If a “short circuit” is formed, the ventilation efficiency will not be as high as the value evaluated by anemometers or funnels, and the virus concentration will not be reduced as much as expected.

Additionally, for example, (2) the method of detecting the concentration of CO2 gas with a sensor or the like and the method of using tracer gas have the following problems.
(2A) The diffusion behavior of CO2 gas and tracer gas, which are gases, is different from that of aerosol viruses, and they are not suitable for evaluating the risk of aerosol infection.
(2B) It is not possible to evaluate the purification ability of air purifiers, etc. through adsorption and dust collection.
(2C) In particular, the method of detecting the concentration of CO: gas cannot be accurately measured in environments where CO: gas other than human breathing is generated, such as when cassette stoves are used in the audience seats.
(2D) Since the concentration of CO: gas emitted by humans is measured, it is not possible to assess the risk of infection when the store is closed with no customers, and it is not possible to assess the risk of infection when the store is occupied by fewer customers than the original capacity. There is a risk of underestimating the risk of infection in the space.

Furthermore, for example, the method (3) of photographing mist particles has the following problems.
(3A) It is not possible to photograph mist particles as an image unless a considerable amount of mist particles are generated, so it cannot be called a simple method.
(3B) For example, in a space with partitions such as a pub, the field of view is obstructed by the partitions, making it impossible to photograph mist particles.
(3C) If mist particles are used, there is a risk that fire alarms (for example, photoelectric spot detectors, etc.) will react, causing malfunctions such as sounding the alarm or closing fire shutters and fire doors.

As described above, the conventional method of evaluating infection risk based on the ventilation capacity of a store has not been able to objectively evaluate the infection risk in a store.

One object of the present inventions is to provide a method for easily evaluating whether there is an aerosol infection risk in a space having a defined volume.

### Solution to Problem

A method for evaluating an aerosol infection risk of the first aspect includes: a release step of releasing a plurality of fine particles having an average particle size of 10 μm or less into a space having a predetermined volume so that the amount in the space is at least 50 mg/m³; an identifying step includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; and, an evaluation step of evaluating that there is an aerosol infection risk in the space when the reduction capacity is less than the standard reduction capacity required for the space.

The method for evaluating the aerosol infection risk of the second aspect is the method of the first aspect, wherein, in the evaluation step, evaluating that there is no aerosol infection risk in the space when the reduction capacity is equal to or higher than the standard reduction capacity.

The method for evaluating the aerosol infection risk of the third aspect is the method of the first or the second aspect, wherein the standard reduction capacity is a required ventilation capacity required for the volume of the space or a modified required ventilation capacity obtained by modifying the required ventilation capacity based on the usage mode of at least one or more users who use the space.

The method for evaluating the aerosol infection risk of the fourth aspect is the method of the third aspect, further includes a preparing step of preparing a report including the reduction capability identified in the identifying step.

The method for evaluating the aerosol infection risk of the fifth aspect is the method of the fourth aspect, wherein the preparing step prepares the report further including information on the two-dimensional spectrum of the relationship.

The method for evaluating the aerosol infection risk of the sixth aspect is the method of the fourth aspect, wherein the preparing step prepares the report further including information regarding the insufficient capacity of the reduction capacity required for evaluating that there is no aerosol infection risk in the space, when evaluating that there is an aerosol infection risk in the space.

The method for evaluating the aerosol infection risk of the seventh aspect is the method of the fourth aspect, wherein the preparing step prepares the report further including information regarding the extra capacity of the reduction capacity required for evaluating that there is not no aerosol infection risk in the space, when evaluating that there is no aerosol infection risk in the space.

The evaluating program for evaluating an aerosol infection risk of one aspect causes a computer to perform; a release function of releasing a plurality of fine particles having an average particle size of 10 μm or less into a space having a predetermined volume so that the amount in the space is at least 50 mg/m³; an identifying function includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of fine particles measured at each elapsed time, and identifying a reduction capacity to reduce a plurality of fine particles in the space from the specific elapsed time; and, an evaluating function of evaluating that there is the aerosol infection risk in the space when the reduction capacity is less than the standard reduction capacity required for the space and evaluating that there is no aerosol infection risk in the space when the reduction capacity is equal to or higher than the standard reduction capacity.

An evaluating apparatus for evaluating an aerosol infection risk of the first aspect includes; an identifying device measuring a plurality of times the number of a plurality of fine particles having an average particle size of 10 μm or less released in a space having a predetermined volume so that the amount in the space is at least 50 mg/m³ or more, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; an evaluating device evaluating that there is an aerosol infection risk in the space when the reduction capacity is less than the standard reduction capacity required for the space and evaluating that there is no aerosol infection risk in the space when the reduction capacity is equal to or higher than the standard reduction capacity; and, a controller controlling the identifying device and the evaluating device based on the evaluating program for evaluating an aerosol infection risk of the one aspect.

An evaluating apparatus for evaluating an aerosol infection risk of the second aspect includes; a release device releasing a plurality of fine particles having an average particle size of 10 μm or less into a space having a predetermined volume so that the amount in the space is at least 50 mg/m³; an identifying device measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; an evaluating device evaluating that there is an aerosol infection risk in the space when the reduction capacity is less than the standard reduction capacity required for the space and evaluating that there is no aerosol infection risk in the space when the reduction capacity is equal to or higher than the standard reduction capacity; and, a controller controlling the identifying device and the evaluating device based on the evaluating program for evaluating an aerosol infection risk of the one aspect.

The evaluating apparatus for evaluating an aerosol infection risk of the third aspect further includes, in the method of the first or the second apparatus, a preparing device preparing evaluation results evaluated by the evaluating device and information accompanying the evaluation results as a report.

An air purification system of the first aspect includes: the evaluating apparatus for evaluating an aerosol infection risk of the second aspect; and, an air purification device having a housing in which an air inlet and an air outlet are formed; an air blowing source for drawing air into the air inlet and exhausting air from the air outlet; and a catcher for catching a plurality of fine particles sucked from the air inlet together with air, the air purification device is controlled by the controller to purify air taken in through the air inlet and exhaust purified air through the air outlet, wherein the air blowing source can adjust the air blowing volume and the controller controls the air blow source and causes the air blow source to blow more air when the evaluating device evaluates that there is an aerosol infection risk in the space.

The air purification system of the second aspect further includes, in the system of the first aspect, a notification device notifying a user, when the evaluating device evaluates there is an aerosol infection risk in the space, there is the aerosol infection risk in the space.

An air purification system of the third aspect includes: the evaluating apparatus for evaluating an aerosol infection risk of the apparatus of the second aspect; and, an air purification device having a housing in which an air inlet and an air outlet are formed; an air blowing source for drawing air into the air inlet and exhausting air from the air outlet; and a catcher for catching a plurality of fine particles sucked from the air inlet together with air, the air purification device is controlled by the controller to purify air taken in through the air inlet and exhaust purified air through the air outlet, wherein the catcher can adjust the amount of catching a plurality of fine particles and the controller controls the catcher and causes the catcher to catch more fine particles when the evaluating device evaluates that there is an aerosol infection risk in the space.

The air purification system of the fourth aspect further includes, in the system of the third aspect, a notification device notifying a user, when the evaluating device evaluates there is an aerosol infection risk in the space, there is the aerosol infection risk in the space.

The air purification system of the fifth aspect is the system of any one of the first to fourth aspect, wherein the air purification device can humidify and/or adjust the temperature of the air that is taken in through the air inlet and then exhausted through the air outlet.

An air purification system of the sixth aspect includes: the evaluating apparatus for evaluating an aerosol infection risk of the second aspect; and, an exhaust out-let exhausting air in the space to the outside, wherein the exhaust out-let can adjust the amount of air exhausted and the controller controls the catcher and causes the exhaust out-let to exhaust more air when the evaluating device evaluates that there is an aerosol infection risk in the space.

The air purification system of the seventh aspect further includes, in the system of the sixth aspect, a notification device notifying a user, when the evaluating device evaluates there is an aerosol infection risk in the space, there is the aerosol infection risk in the space.

A method for evaluating a dust damage risk of the first aspect includes: a release step of releasing a plurality of fine particles having an average particle size of 10 μm or less into a space having a predetermined volume so that the amount in the space is at least 50 mg/m³; an identifying step includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; and, an evaluation step of evaluating that there is an aerosol infection risk in the space when the reduction capacity is less than the standard reduction capacity required for the space.

An evaluating program for evaluating a dust damage risk of the first aspect causes a computer to perform; a release function of releasing a plurality of fine particles having an average particle size of 10 μm or less into a space having a predetermined volume so that the amount in the space is at least 50 mg/m³; an identifying function includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of fine particles measured at each elapsed time, and identifying a reduction capacity to reduce a plurality of fine particles in the space from the specific elapsed time; and, an evaluating function of evaluating that there is no aerosol infection risk in the space when the reduction capacity is equal to or higher than the standard reduction capacity.

An evaluating apparatus for evaluating a dust damage risk of the first aspect includes; an identifying device measuring a plurality of times the number of a plurality of fine particles having an average particle size of 10 μm or less released in a space having a predetermined volume so that the amount in the space is at least 50 mg/m³ or more, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; an evaluating device evaluating that there is an aerosol infection risk in the space when the reduction capacity is less than the standard reduction capacity required for the space and evaluating that there is no aerosol infection risk in the space when the reduction capacity is equal to or higher than the standard reduction capacity; and, a controller controlling the identifying device and the evaluating device based on the evaluating program for evaluating an aerosol infection risk according to the one aspect.

An evaluating method for evaluating a dust damage risk of the fourth aspect includes;
in a space having a predetermined volume, (1) the liquid particles are composed of a volatile organic compound, (2) the average particle size is 10 μm or less, and (3) the number of liquid particles released into the space is volatile, a discharge step of discharging a plurality of liquid fine particles in a quantity corresponding to a quantity less than the saturated vapor quantity of the organic compound; after the discharge step, the quantity of a plurality of liquid particles is measured multiple times in the space, and from the relationship between the elapsed time from the end of the discharge step and the number of the plurality of liquid particles measured, Deriving a specific elapsed time until the number of liquid particles decreases to a predetermined number, and specifying a reduction ability of a plurality of liquid particles in the space from the specific elapsed time;
an evaluation step of evaluating that there is a risk of aerosol infection in the space if the reduction capacity is less than the standard reduction capacity required for the space.

The aerosol infection risk assessment device according to the fifth aspect includes:
in the aerosol infection risk assessment device of the fourth aspect, in the evaluation step, if the reduction ability is equal to or higher than the reference reduction ability, it is evaluated that there is no risk of aerosol infection within the space.

The aerosol infection risk assessment program according to the second aspect includes;
the program causes the computer to perform the following functions:
in a space having a predetermined volume, (1) the liquid particles are composed of a volatile organic compound, (2) the average particle size is 10 μm or less, and (3) the number of liquid particles released into the space is volatile, a release function that releases a plurality of liquid particles in an amount corresponding to an amount less than the saturated vapor amount of the organic compound; after the discharge of the plurality of liquid particles is completed, the quantity of the plurality of liquid particles is measured multiple times in the space, and the number of the plurality of liquid particles is measured as the elapsed time from the end of the discharge of the plurality of liquid particles. Deriving a specific elapsed time until the number of a plurality of liquid particles decreases to a predetermined number from the relationship with the number of liquid particles in the space, and reducing the plurality of liquid particles in the space from the specific elapsed time, a specific function that specifies the ability;
if the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation function that evaluates that there is no risk of aerosol infection in.

The aerosol infection risk assessment device according to the fifth aspect includes:
in a space having a defined volume, (1) is composed of a volatile organic compound, (2) has an average particle size of 10 μm or less, and (3) has a quantity equivalent to less than the saturated vapor amount of the volatile organic compound in the space. The quantity of the plurality of liquid particles discharged into the space is measured multiple times, and the elapsed time from the end of the discharge of the plurality of liquid particles is calculated from the number of the plurality of liquid particles to be measured, a specifying unit that derives a specific elapsed time until the number of the plurality of liquid particles decreases to a predetermined amount from the relationship, and specifies a reduction ability of the plurality of liquid particles in the space from the specific elapsed time;
if the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation department that assesses that there is no risk of aerosol infection at a control unit that controls the identification unit and the evaluation unit based on the aerosol infection risk assessment program according to claim 8.

### Advantageous Effects of Invention

The method for evaluating an aerosol infection risk of the first aspect can easily evaluate whether there is an aerosol infection risk in a space with a defined volume.

The method for evaluating an aerosol infection risk of the second aspect can easily evaluate whether there is an aerosol infection risk in a space with a defined volume.

The method for evaluating an aerosol infection risk of the third aspect can inform the reduction ability by the generated report.

The method for evaluating an aerosol infection risk of the fourth aspect can provide a report that visualizes the relationship between the elapsed time from the end of the process of releasing a plurality of fine particles and the number of the measured plurality of fine particles.

The method for evaluating an aerosol infection risk of the fifth aspect can inform the lack of reduction ability in the space when evaluating that there is an aerosol infection risk in the space.

The method for evaluating an aerosol infection risk of the sixth aspect can inform the extra capacity of the reduction capacity in the space when evaluating that there is no aerosol infection risk in the space.

The evaluating program for evaluating a dust damage risk of one aspect can cause a computer to easily evaluate whether there is an aerosol infection risk in a space having a defined volume.

The evaluating apparatuses for evaluating an aerosol infection risk of the first and second aspects can easily assess whether there is an aerosol infection risk in a space having a defined volume.

The evaluating apparatus for evaluating an aerosol infection risk of the third aspect can create a report including whether there is an aerosol infection risk in a space having a defined volume.

The air purification systems of the first, third and sixth aspects can automatically reduce the risk of aerosol infection when evaluating that there is an aerosol infection risk in the space.

The air purification systems of the second, fourth and seventh aspects can inform the user of the aerosol infection risk in the space when it is evaluated.

The air purification system of the fifth aspect can exhibit one or both humidifying function and the temperature adjusting function as well as the air cleaning function that reduces the aerosol infection risk.

One embodiment of the dust damage risk evaluation method can easily evaluate whether there is a dust damage risk in a space having a defined volume.

One aspect of the dust damage risk evaluation program can cause a computer to easily evaluate whether there is a dust damage risk in a space having a predetermined volume.

The dust damage risk evaluation device of one embodiment can easily evaluate whether there is a dust damage risk in a space having a predetermined volume.

The aerosol infection risk evaluation method of the fourth aspect can easily and simply evaluate whether there is an aerosol infection risk within a space having a defined volume.

The aerosol infection risk evaluation method of the fifth aspect can easily and simply evaluate whether there is an aerosol infection risk within a space having a defined volume.

The aerosol infection risk evaluation program of the second aspect allows a computer to easily and simply evaluate whether or not there is an aerosol infection risk within a space having a defined volume.

The aerosol infection risk evaluation device of the fifth aspect can easily and simply evaluate whether there is an aerosol infection risk within a space having a defined volume.

### BRIEF DESCRIPTION OF DRAWINGS

FIG. 1 is a plan view of the laboratory used in Examples 1 to 27.

FIG. 2 is a side view of the laboratory in FIG. 1.

FIG. 3 is a graph showing the results of evaluation experiments of Examples 1 to 6.

FIG. 4 is a graph showing the results of evaluation experiments for Examples 7 to 12.

FIG. 5 is a graph showing the results of evaluation experiments for Examples 13 to 22.

FIG. 6 is a graph showing the results of evaluation experiments for Examples 23 to 25.

FIG. 7 is a graph showing the results of Examples 26 and 27.

FIG. 8 is a plan view of the hole used in Examples 28 to 32.

FIG. 9 is another plan view of the hole in FIG. 8.

FIG. 10 is a graph showing the results of evaluation experiments for Examples 28 to 32.

FIG. 11 is a plan view of a semi-private room used in Examples 33-34.

FIG. 12 is a front view of the wall of the semi-private room in FIG. 11.

FIG. 13 is a front view of another wall of the semi-private room of FIG. 11.

FIG. 14 is a front view of yet another wall of the semi-private room of FIG. 11.

FIG. 15 is a graph showing the results of evaluation experiments for Examples 33 to 37.

FIG. 16 is a plan view of a semi-private room used in Examples 35 to 37.

FIG. 17 is a plan view of the semi-private room shown in FIG. 16.

FIG. 18 is a front view of the wall of the semi-private room shown in FIG. 16.

FIG. 19 is a plan view of a store including semi-private rooms shown in FIGS. 11 and 16.

FIG. 20 is Table 1 of Examples.

FIG. 21 is Table 2 of Examples.

FIG. 22 is Table 3 of Examples.

FIG. 23 is Table 4 of Examples.

FIG. 24 is Table 5 of Examples.

FIG. 25 is Table 6 of Examples.

FIG. 26 is Table 7 of Examples.

FIG. 27 is Table 8 of Examples.

FIG. 28 is Table 9 of Examples.

FIG. 29 is Table 10 of Examples.

FIG. 30 is a block diagram of the evaluating apparatus of this embodiment.

FIG. 31 is a flow chart of the method for evaluating an aerosol infection risk performed using the evaluating apparatus of this embodiment.

FIG. 32 is a flow chart of the method for evaluating an aerosol infection risk of HACCP-type aerosol infection, which is performed using the modified evaluating apparatus.

FIG. 33 is a block diagram of the air purification system of this embodiment.

FIG. 34 is a schematic diagram of the air purification apparatus of the present embodiment.

FIG. 35 is a flowchart of the method for evaluating an aerosol infection risk performed using the air purification system of the present embodiment;

### DESCRIPTION OF EMBODIMENTS

### Embodiment

Hereinafter, embodiments of the present invention (hereinafter referred to as the present embodiments) will be described in the following order of description.
1. How to assess the risk of aerosol infection
2. How to reduce the risk of aerosol infection
3. Example
4. Summary and supplements of 1 to 3
5. Aerosol infection risk assessment device and assessment program
6. HACCP type aerosol infection risk evaluation device and evaluation program
7. air purification system
8. Variations
9. Supplementary information regarding 1-3 and 5-8

Here, this specification has been created in two parts. Specifically, the above 1-3 was written in the first period, and 4-8 was written in the second period, which is after the first period. The content written in the second period was created by reviewing the content written in the first period and adding specific hardware.

### «1. How to Assess the Risk of Aerosol Infection»

First, the aerosol infection risk evaluation method of this embodiment (hereinafter referred to as the evaluation method of this embodiment) will be described with reference to the drawings. The evaluation method of this embodiment was created through intensive study by the inventor of the present application to solve the above-mentioned problem.

<Summary of Evaluation Method of this Embodiment>

The evaluation method of this embodiment includes the following (1) to (3).
(1) Using multiple fine particles
(2) Based on the recovery time t1 (an example of a specific elapsed time), which is the time required for the number of the plurality of particles to sufficiently decrease, the particles in the gathering space (an example of a space having a defined volume) Evaluating the removal ability (or “ability to reduce multiple particulates”)
(3) Compare the particulate removal capacity with the required ventilation capacity of the gathering space.

According to the evaluation method of this embodiment, the behavior of droplet nuclei in an aerosol can be simulated by the behavior of fine particles, and the actual droplet nucleus removal ability of the customer space, which is difficult to evaluate based on the ventilation capacity of the facility alone, can be simulated. It can be easily evaluated. Furthermore, compared to methods that use tracer gas, fine particles imitating droplet nuclei can be visually observed, making it easier to understand the actual flow and diffusion of droplet nuclei.

<Details of Evaluation Method of this Embodiment>

Next, the evaluation method of this embodiment will be specifically described with reference to the drawings.

As an example, the evaluation method of this embodiment evaluates the risk of infection by an infectious disease in a gathering space inside a facility.

### [Definition, etc.]

Hereinafter, an example of definitions of multiple terms used in this specification will be explained.

“Infection risk” literally means the risk of contracting an infectious disease. In this embodiment, it refers to the risk of aerosol infection caused by aerosols containing pathogens.

“Infectious disease” means a disease caused by a pathogen invading the body, and “pathogen” means a pathogenic microorganism (causing an infectious disease). Examples of “pathogens” are viruses, bacteria, fungi, parasites, and the like.

“Aerosol” means a sol in which a plurality of fine particles are dispersed in air, which is a dispersion medium. Moreover, “aerosol infection” means, for example, infection mediated by droplet nuclei (microdroplets) containing a pathogen and having a particle diameter of 10 μm or less.

“Aerosol infection” is a different concept from “droplet infection.” “Droplet infection” is an infection that occurs through droplet particles containing pathogens and water when an infected person or carrier coughs or sneezes. Since droplet particles are relatively large in size (more than 10 μm) and heavy, pathogens are said to be dispersed only within a range of approximately 1 m to 2 m.

On the other hand, “aerosol infection” is an infection that occurs through droplet nuclei that are generated when water evaporates from droplet particles. Since droplet nuclei are relatively small and light particles, they float in the air for a long time and are said to spread over a wider area than droplet particles, causing clusters to form. There is.

The evaluation method of this embodiment is a method for evaluating the risk of aerosol infection of the new coronavirus infection (COVID-19) in gathering spaces, where the infection risk is the risk of aerosol infection caused by aerosols containing the new coronavirus. Suitably used.

The evaluation method of this embodiment includes, as an example, (1) a step of discharging a plurality of liquid particles into a gathering space, (2) a step of tracking the quantity of a plurality of liquid particles, and (3) a particle removal ability of the gathering space, and (4) assessing the risk of aerosol infection in gathering spaces. Each of the steps (1) to (4) will be explained below in the order in which they are described.

[(1) Step of Releasing Multiple Liquid Particles into the Gathering Space]

In this step, a plurality of liquid microparticles are released into the gathering space using a release source (not shown). It is preferable that the average particle diameter of the plurality of liquid particles emitted from the emission source is, for example, 10 μm or less. This is to understand the behavior of droplet nuclei by using liquid microparticles that have the same average particle size as the droplet nuclei (microdroplets) that mediate aerosol infection. Note that the “average particle diameter” in this specification is, for example, the average particle diameter measured by a laser light scattering method.

The components of each of the liquid particles constituting the plurality of liquid particles are not particularly limited. The components of each liquid microparticle may include, for example, ethylene glycol and water. Examples of the chemical solution that serves as the stock solution of each liquid fine particle include the trade name “FLG5 Heavy Type” (manufactured by ANTARI).

The source of the plurality of liquid particles is not particularly limited either. The release source may have, for example, a mechanism for heating a chemical liquid that is a stock solution of a plurality of liquid particles with a heater and vaporizing the chemical liquid. An example of a release source is a commercially available smoke machine (such as a stage production smoke machine). The type of smoke machine is also not particularly limited. For example, there is a product name “Z800II Fog Machine” (manufactured by ANTARI).

When the release source releases a plurality of liquid particles into the space to be evaluated, it is preferable that the release amount is, for example, 50 mg/m 3 or more. This reduces the influence of the number of other particles that normally exist within the space to be released (collection space), and allows the measurement of multiple liquid particles released from the emission source into the space (collection space). It is possible to improve the S/N ratio during measurement using a particle measuring device (to be described later).

### [(2) Tracking Process of Quantity of Multiple Liquid Particles]

(1) After the step of releasing a plurality of liquid particles into the gathering space, in this step, the quantity of the plurality of liquid particles released into the gathering space is measured over time. Here, the means for measuring the number of liquid particles is not particularly limited. Measurement means include, for example, a densitometer that measures the concentration of particulates based on a gravimetric method (filter weighing method), a particulate counter (particle counter) that measures the number of multiple particulates from the relative concentration calculated by a laser light scattering method, etc. It is a measuring device.

Compared to the measurement method using a densitometer, the measurement method using a particle counter can measure the quantity of multiple liquid particles without being affected by the average particle diameter. Therefore, the measurement method using a particulate meter is more accurate in time-based measurement of the quantity of a plurality of liquid particles changing in the air than the measurement method using a densitometer. Examples of particulate measuring instruments include the product name “DT-9880” (manufactured by CEM).

When measuring the number of a plurality of liquid particles in a time-based manner, for example, it is preferable to measure the number of liquid particles with an average particle diameter of 0.3 μm or more and 10 μm or less. The reason for this is to track the behavior of liquid microparticles that have the same average particle size as the droplet nuclei (microdroplets) that mediate aerosol infection, and to understand the behavior of the droplet nuclei.

In addition, when measuring the quantity of multiple liquid microparticles time-wise, the release process is It is preferable that the number of the plurality of fine particles existing in the collection space is measured before the measurement, and the difference between the number and the measured number is taken as the number of the plurality of liquid fine particles.

### [(3) Evaluation Process of Particulate Removal Ability of Gathering Space]

In this step, during or after the step of (2) tracking the number of liquid particles, aggregation is performed based on the recovery time t1, which is the time required until the number of liquid particles is sufficiently reduced. Evaluate the particulate removal capacity of the space.

“Particle removal ability” means the ability to remove multiple liquid particles from the air in the gathering space to be evaluated. “Particle removal ability” is a different concept from “ventilation ability.” “Ventilation capacity” means the ability to discharge a plurality of liquid particles from inside the gathering space to the outside through a ventilation system such as a ventilation fan installed on the wall of the gathering space, for example.

On the other hand, “particulate removal ability” includes, for example, removal based on emissions from ventilation systems, removal based on emissions from openings in gathering spaces such as windows, air purifiers, air conditioners, etc. Includes capabilities such as air agitation, decomposition of multiple liquid particles, and removal based on adsorption. That is, in this specification, “particle removal ability of a gathering space, which is an example of an evaluation target” means the ability to remove a plurality of liquid particles based on the configuration of the gathering space. Note that “fine particle removal ability” has the same meaning as the ability to reduce a plurality of liquid fine particles, which will be described later. These abilities are evaluated more highly as the recovery time t1 is smaller.

“Sufficiently reduced” means that the number of liquid particles released into the air in the gathering space to be evaluated has decreased, so that the number of liquid particles in the air in the gathering space to be evaluated has decreased. This means that the number of microparticles present in the microparticles is comparable to the number of microparticles present before the release step.

For example, the “time required to sufficiently reduce the amount” is the amount of liquid particles that is 1/100 of the maximum number of liquid particles present in the air in the gathering space after the discharge process. It corresponds to the elapsed time until the quantity decreases.

Regarding reducing the amount to 1/100 when the recovery time t1 is specified, it is assumed that there is no risk of infection if the amount of virus released into the air is diluted to 1/100 of the maximum value or less. There is an editorial (advocated by Takayuki Miyazawa, Associate Professor, Department of Viral Coevolution, Institute for Frontier Medical Sciences, Kyoto University). Even if a virus exists, it is said that a person will not be infected unless a certain amount of the virus is taken into the body.

If the recovery time t1 is specified by one tenth of the quantity, there is a possibility that the recovery time t1 will vary depending on the average particle diameter of the plurality of liquid particles. For this reason, to accurately determine the particulate removal capacity, the maximum number of liquid particulates present in the air of the gathering space after the discharge process must be reduced to one-hundredth of the maximum number. It is preferable that the specification is determined by the elapsed time.

### [(4) Assessment Process of Aerosol Infection Risk in Gathering Spaces]

As an example, this step is a step performed during or after the step (3) evaluating the particulate removal capacity of the gathering space, and compares the particulate removal capacity with the required ventilation capacity of the gathering space, and compares the particulate removal capacity with the required ventilation capacity of the gathering space. The gathering space is evaluated as having a low risk of aerosol infection (or no risk of aerosol infection) if the ventilation capacity is equal to or greater than the required ventilation capacity.

Here, as an example, the specific method of evaluation in this step is as follows.

### < (4A) First Infection Risk Assessment Method>

The first infection risk assessment method compares the particulate removal amount Q1 and the required human ventilation amount Q2, and if the particulate removal amount Q1 is equal to or higher than the required human ventilation amount Q2, the risk of aerosol infection in the gathering space is low. (or there is no risk of aerosol infection), and if the amount of particulate removal Q1 is less than the required human ventilation amount Q2, it is evaluated that the risk of aerosol infection in the gathering space is high (or there is a risk of aerosol infection).

In the first infection risk evaluation method, the particulate removal ability is calculated from the following formula (1). The particulate removal amount Q1 is the number of liquid particulates per 1 [m³] that are removed in 60 [minutes] in the gathering space to be evaluated. Since the recovery time t1 can be regarded as the time required to remove all the liquid particles released into the gathering space and filling the gathering space, the number of particles removed Q1 is determined by the volume V of the gathering space and the recovery time. It can be calculated from t1.

$$Particulate removal amount Q 1 [ {m}^{3} / hr . ] = Volume of gathering space V [ {m}^{3} ] \times 60 [ minutes ] / Recovery time t 1 [ minutes ] \qquad Formula ( 1 )$$

In addition, in the first infection risk assessment method, the required ventilation amount is defined by the required human ventilation amount Q2 calculated by the following formula (2). The required human ventilation amount Q2 is the ventilation amount required for the gathering space to be measured according to its accommodating capacity, and is calculated from the required ventilation amount per customer and the accommodating capacity n of the gathering space.

$$Required ventilation amount Q 2 [ {m}^{3} / hr . ] = Required ventilation amount per customer [ {m}^{3} / hr . / person ] \times Number of occupancy of gathering space n [ person ] \qquad Formula ( 2 )$$

According to the first infection risk evaluation method, the infection risk can be evaluated more accurately by taking into consideration, for example, the volume of the gathering space, the number of people it can accommodate, etc. Depending on ventilation, etc., it may not be possible to ensure enough particulate removal, and measures may be taken to reduce the number of occupancy n, but according to the first infection risk assessment method, for example, the influence of the accommodation capacity n, etc. The evaluation will take into consideration.

In Japan, the required ventilation amount per customer can be appropriately adopted from among the values of the required ventilation amount stipulated by the relevant laws and regulations. For example, 20 [m³/hr.] (value based on Article 20-2 of the Enforcement Order of the Building Standards Act), 30 [m³/hr.] (based on the environmental hygiene standards of the Act on Ensuring Sanitary Environments in Buildings), (values that the Ministry of Health, Labor, and Welfare has indicated are necessary from the perspective of coronavirus countermeasures). The standards based on these laws and regulations are values determined by environmental health experts from the perspective of preventing infectious diseases, and are reliable values.

Below, for reference, an example of the calculation method based on the law (Article 20-2 of the Enforcement Order of the Building Standards Act) is shown.

The effective ventilation volume (equivalent to “required ventilation volume” in this specification) shall be greater than or equal to the value calculated using the following formula.

$$V = 20 Af / N \qquad ( Formula )$$

In this formula, V, Af, and N each represent the following numerical values.
V: Effective ventilation volume [pcs/hr./m³]
Af: Floor area of the living room [m²] (If a living room other than the living room of a special building has a window or other opening that is effective for ventilation, it is obtained by multiplying the area that is effective for ventilation of the opening by 20. (area obtained by subtracting the area from the floor area of the living room)
N: Occupied area per person according to actual conditions [m²] (For rooms in special buildings, if it exceeds 3, set it as 3; for other rooms, if it exceeds 10, set it as 10.)

### <(4B) Second Infection Risk Assessment Method>

The second infection risk assessment method compares the number of particulate removal times N1 with the required number of ventilations N2, and if the number of particulate removals N1 is equal to or greater than the required number of ventilations N2, the risk of aerosol infection in the gathering space is low (or If the number of times N1 of particulate removal is less than the required number of ventilations N2, the risk of aerosol infection in the gathering space is high (or there is a risk of aerosol infection).

In the second infection risk evaluation method, the particulate removal ability is evaluated by the number of particulate removal times N1 calculated by the following formula (3). The particle removal number N1 indicates how many times per hour all liquid particles discharged and filling the gathering space to be evaluated can be removed. Since the recovery time t1 can be regarded as the time required to remove all the liquid particles released into the gathering space and filling the gathering space, the number of times N1 of particle removal can be calculated from the recovery time t1, can.

$$Number of particulate removal times N 1 [ times / hr . ] = 60 [ minutes ] / Recovery time t 1 [ minutes ] \qquad Formula ( 3 )$$

In addition, in the second infection risk assessment method, the required ventilation amount is defined by the required ventilation frequency N2 of the gathering space, which is determined in accordance with the guidelines of the Institute of Hygiene. The required ventilation frequency N2 is the ventilation frequency determined according to the business type and purpose of use of the gathering space to be evaluated, and it stipulates how many times the air filling the gathering space should be ventilated (exchanged) per hour. It is. For example, in the case of cafeterias and restaurants, the required ventilation frequency is specified as 6 [times/hr.].

The second infection risk assessment method is effective in that even if the volume of the gathering space, the number of people it can accommodate, etc. are unclear, the infection risk can be easily assessed as long as the business type and purpose of use are known. However, the guidelines for sanitary laboratories are based on the average occupancy density based on industry type and purpose of use, so if you want to perform a more accurate assessment, it is preferable to use the first infection risk assessment method.

### <(4C) Third Infection Risk Assessment Method>

The third infection risk assessment method compares the recovery time t1 and the required ventilation time t2, and if the recovery time t1 is equal to or greater than the required ventilation time t2, the aerosol infection risk in the gathering space is low (or the aerosol infection risk is low). If the recovery time t1 is less than the required ventilation time t2, it is evaluated that the risk of aerosol infection in the gathering space is high (or there is a risk of aerosol infection).

In the third infection risk evaluation method, the ability to remove particulates is determined from the recovery time t1. Further, in the third infection risk evaluation method, the required ventilation amount is specified by the required ventilation time t2 calculated by the following equation (4). The required ventilation time t2 defines the maximum time required to remove the air filling the gathering space to be evaluated. The required ventilation time t2 can be calculated from the required ventilation frequency N2.

$$Required ventilation time t 2 [ minutes ] = 60 [ minutes ] / Required number of ventilations N 2 [ times / hr . ] \qquad Formula ( 4 )$$

The third infection risk assessment method, like the second infection risk assessment method, can easily assess the infection risk even if the volume and capacity of the gathering space are unclear, if the business type and purpose of use are known. It is effective in that it allows evaluation of However, the guidelines for sanitary laboratories are based on the average occupancy density based on industry type and purpose of use, so if you want to perform a more accurate assessment, it is preferable to use the first infection risk assessment method.

### <(4D) Fourth Infection Risk Assessment Method>

The fourth infection risk assessment method compares the particulate removal amount Q1 with the area required ventilation amount Q3, and if the particulate removal amount Q1 is equal to or greater than the area required ventilation amount Q3, the risk of aerosol infection in the gathering space is low. (or there is no risk of aerosol infection), and if the particulate removal amount Q1 is less than the area required ventilation amount Q3, it is evaluated that the aerosol infection risk in the gathering space is high (or there is a risk of aerosol infection).

In the fourth infection risk evaluation method, the particulate removal ability is calculated from equation (1) of the first infection risk evaluation method. Further, in the fourth infection risk evaluation method, the required ventilation amount is defined by the area required ventilation amount Q3 calculated from the following equation (5). While the required human ventilation Q2 used in the first infection risk assessment method was determined based on the volume V of the gathering space, the required area ventilation Q3 was determined based on the floor area of the gathering space. This is the ventilation amount.

$$Area required ventilation amount Q 3 [ m 3 / hr . ] = Required ventilation amount per floor area [ {m}^{3} / {m}^{2} / hr . ] \times Floor area of gathering space [ {m}^{2} ] \qquad Formula ( 5 )$$

The area required ventilation amount Q3 can be calculated from the required ventilation amount per floor area and the floor area of the gathering space. Here, the floor area required ventilation amount is a value specified in the Standards of the Society of Air Conditioning and Sanitary Engineers (HASS 102, 1972). The amount of ventilation required for the floor space is determined depending on the type of business and purpose of use of the gathering space to be evaluated. For example, in the case of cafeterias and restaurants, the required floor space ventilation rate is 30 [m³/m2/hr.] for “restaurants/cafes (normal)” and 17.7 [m³/m²/hr.] for “restaurants/cafes (luxury)”.

The fourth infection risk assessment method is that even if the volume of the gathering space or the number of people it can accommodate is unclear, the risk of infection can be easily assessed if the type of business, purpose of use, and floor area of the space are known. It is valid in points. The floor area is stated in, for example, the business license of a restaurant business, and there is a high possibility that the business owner knows this. However, the guidelines for sanitary laboratories are based on the average occupancy density based on business type and purpose of use, so if you want to perform a more accurate assessment, it is preferable to use the first infection risk assessment method.

### «2. How to Reduce the Risk of Aerosol Infection»

The infection risk reduction method of the present embodiment is for reducing the infection risk of an infectious disease in a gathering space. Specifically, because of performing the infection risk evaluation method of this embodiment, if it is evaluated that the aerosol infection risk is high in the gathering space to be evaluated, the risk of aerosol infection in the gathering space is reduced to a low level (for example, The following improvement measures will be taken until the particulate removal capacity is equal to or higher than the required ventilation capacity.

Specific examples of improvement measures include improving the capacity of ventilation systems such as ventilation fans, ventilating by opening windows, doors, or vents, operating air conditioners, circulators, or air purifiers, installing shielding boards or bulkheads, and housing. Examples include implementing at least one measure selected from the group consisting of reducing capacity. These improvement measures can be suitably used to reduce the risk of infection in facilities where ventilation systems cannot be strengthened for some reason.

By the way, the measures to prevent the spread of the new coronavirus that have been implemented in Japan so far include detecting people infected with the new coronavirus through PCR tests or antigen tests, and identifying the facility based on the infected person's behavioral history (This has been done by restricting the use of facilities similar in size, structure, and other characteristics to facilities where clusters have occurred (active epidemiological investigations). However, there is little scientific basis for regulating the use of similar facilities, and it is unclear whether they are effective or not. The infection prevention measures are causing confusion in Japanese society and are causing excessive decline in Japan's economic activity.

In contrast, the infection risk evaluation method of this embodiment evaluates the environment of a specific space, regardless of the presence or absence of actual infection, and objectively determines whether the space has taken infection prevention measures. It is effective in that it can be evaluated and provide a sense of security to facility users. Furthermore, the infection risk reduction method of this embodiment is performed based on the results of the infection risk evaluation method of this embodiment, and is therefore effective in that it is possible to appropriately reduce the infection risk.

### 3. Example

Examples of the infection risk evaluation method and infection risk reduction method of this embodiment will be described below.

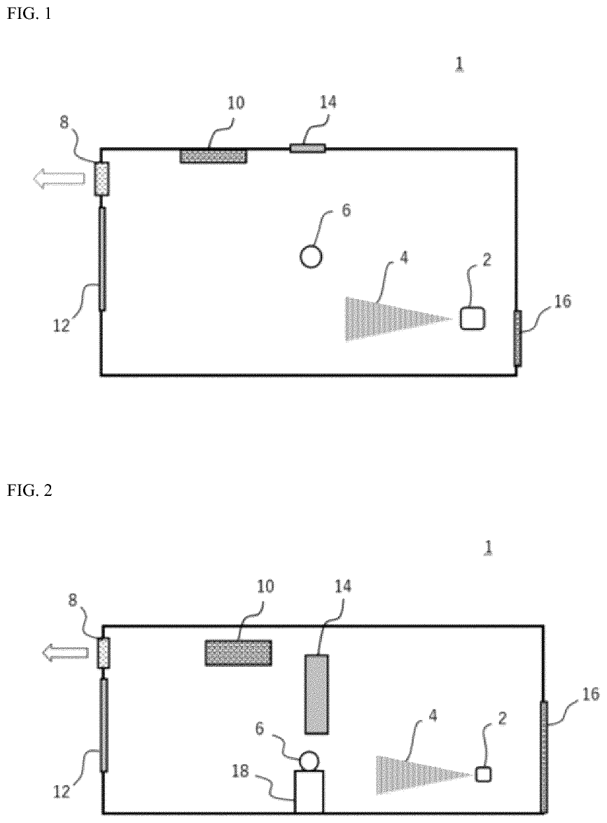

Evaluation of Example 1-27 was conducted in laboratory 1 shown in FIGS. 1 and 2. Laboratory 1 was assumed to be a restaurant. Laboratory 1 is a room measuring 2.6 m long (vertical direction in FIG. 1)×3.0 m wide (horizontal direction in FIG. 1)×2.4 m high, with a floor area of 7.8 m² and a volume V of 18.5 m³.

The accommodating capacity n of laboratory room 1 was 6 people, and the required ventilation amount per customer was 30 [m³/hr.]. From these values and equation (2), the required human ventilation Q2 [m³/hr.] is calculated as 180 [m³/hr.]. In addition, Laboratory 1 falls under the category of “cafeteria, restaurant, and sushi restaurant” in the hypothetical sanitary laboratory guidelines, and the required number of ventilations N2 is 6 times. From this value and equation (4), the required ventilation time t2 [minutes] is calculated as 10 [minutes].

Furthermore, Laboratory 1 falls under the category of “restaurant/coffee (high class)” according to the Air Conditioning and Sanitary Engineers Society Standards (HASS 102 1972), and the required ventilation rate per floor area is 17.7 [m³/m²/hr.], is 7.8 m². From these values and equation (5), the area required ventilation Q3 [m³/hr.] is calculated as 138 [m³/hr.].

The required human ventilation amount Q2, the required number of ventilations N2, the required ventilation time t2, and the required area ventilation amount Q3 in the laboratory 1 under the above conditions are as shown in Table 1 of FIG. Compare these values with the measured values to find the relationship: particulate removal amount Q1=required ventilation amount Q2 (or required area ventilation amount Q3), number of particulate removals N1=required number of ventilations N2, and recovery time t1=required ventilation time t2. If the criteria are met, the risk of infection is low; if not, the risk of infection is assessed to be high.

The risk of infection is evaluated by ejecting liquid particles 4 from a smoke machine 2 in the laboratory 1 shown in FIGS. 1 and 2, tracking the decrease in the number of liquid particles 4 using a particle counter 6, The particle removal ability was evaluated based on the time required for the amount to sufficiently decrease (recovery time).

In addition, in FIGS. 1 and 2, numeral 8 indicates a ventilation fan, numeral 10 indicates an air conditioner, numerals 12 and 14 indicate windows, numeral 16 indicates a door, and numeral 18 indicates an installation stand.

In the examples, the smoke machine used was the product name “Z800II Fog Machine” (manufactured by ANTARI), the chemical liquid used as the liquid particulate stock solution was the product name “FLG5 Heavy Type” (manufactured by ANTARI), and the particle counter was used. The product name “DT-9880” (manufactured by CEM) was used.

### [Effect of Particle Size of Liquid Particles]

### Example 1

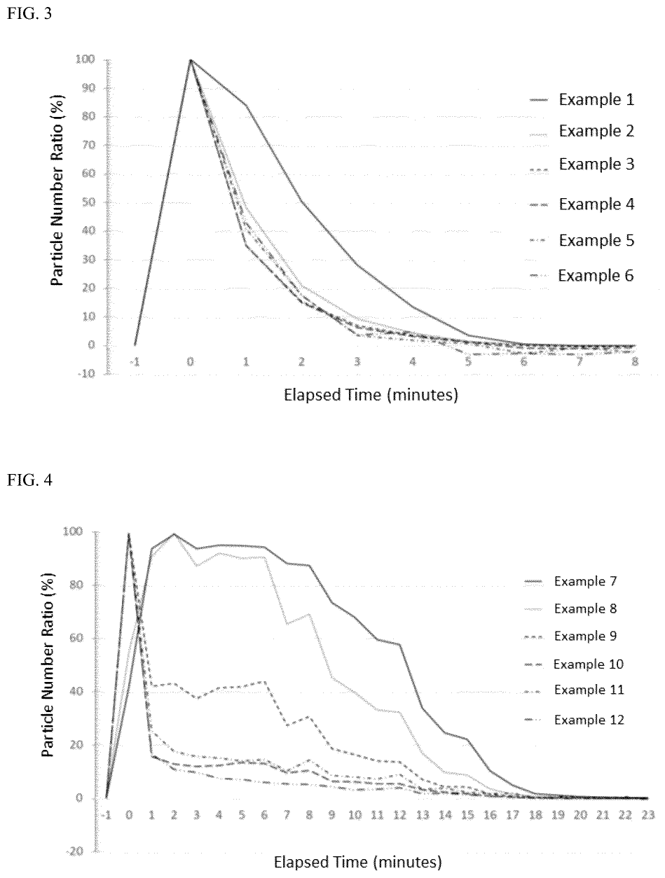

In the laboratory 1 shown in FIGS. 1 and 2, 300 mg/m³ of liquid particles 4 were discharged from the smoke machine 2, and the decrease in the number of liquid particles 4 with a particle diameter of 0.3 μm was tracked using the particle counter 6. To simulate a gathering space with sufficient ventilation, the ventilation volume of the ventilation fan 8 was set to 1000 m³/hr. The evaluation results are shown in Table 2 of FIG. 21 and FIG. 3. Note that the graph shown in FIG. 3 is a graph that tracks the decrease in the number of liquid particles. In this graph, the number of liquid particles at the time of measurement is plotted as a percentage of the maximum number of particles after the liquid particles are discharged (the same applies to graphs shown in subsequent drawings).

### Examples 2-6

The decrease in the number of liquid particles was tracked in the same manner as in Example 1, except that the particle diameter of the liquid particles to be detected was changed to the values listed in Table 2. The results are shown in Table 2 of FIG. 21 and FIG. 3.

### Examples 7-12

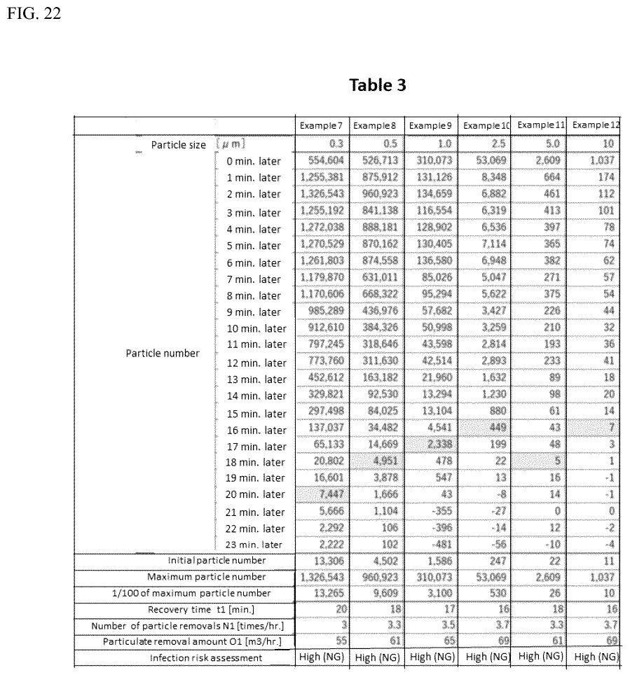

The number of liquid particles was adjusted in the same manner as in Example 1-6, except that the ventilation volume of the ventilation fan 8 shown in FIGS. 1 and 2 was set to 400 m³/hour to simulate a gathering space with insufficient ventilation. The decline was tracked. The results are shown in Table 3 of FIG. 22 and FIG. 4.

Under the conditions of sufficient ventilation in Examples 1-6, the risk of infection was evaluated to be low regardless of the particle size of the liquid particles. Furthermore, even under the conditions of insufficient ventilation in Examples 7-12, the risk of infection was evaluated to be high regardless of the particle size of the liquid particles. In other words, similar evaluation results could be obtained no matter which particle size liquid particles were tracked.

However, as far as the data of Examples 1-12 are concerned, the recovery time t1 tends to become shorter as the particle size increases. This is thought to be since as the particle size of the liquid particles increases, the liquid particles settle and are removed from the space. Therefore, it can be said that more accurate evaluation can be made by reducing the particle size. In this respect, it can be said that it is preferable to track liquid fine particles with a particle size of 0.3 μm or more and 2.5 μm or less, and it can be said that it is preferable to track liquid fine particles with a particle size of 0.3 μm or more and 0.5 μm or less.

### [Effect of the Number of Liquid Particles Released]

### Example 13

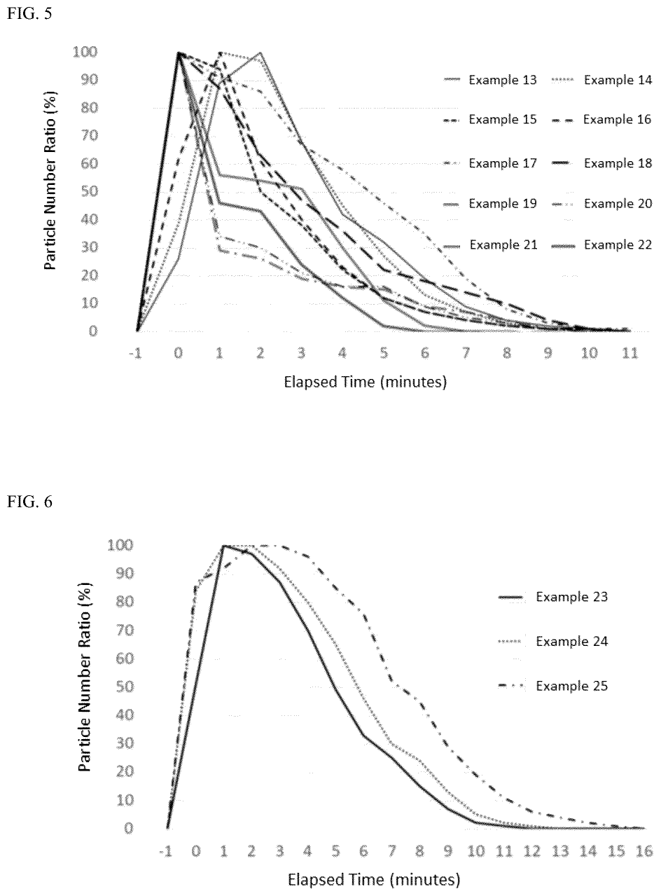

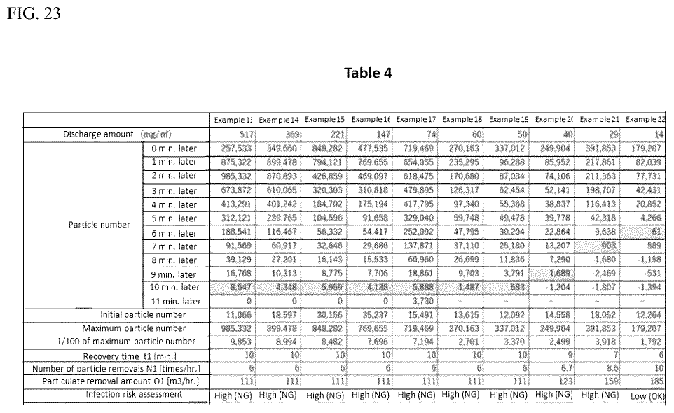

In the laboratory 1 shown in FIGS. 1 and 2, 517 mg/m³ of liquid particles 4 were discharged from the smoke machine 2, and the decrease in the number of liquid particles 4 with a particle diameter of 0.3 μm was tracked using the particle counter 6. Note that the ventilation volume of the ventilation fan 8 was set to 600 m³/hour. The evaluation results are shown in Table 4 of FIG. 23 and FIG. 5.

### Examples 14-22

The decrease in the number of liquid particles was tracked in the same manner as in Example 13, except that the number of liquid particles released was changed to the value shown in Table 4. The results are shown in Table 4 of FIG. 23 and FIG. 5.

In Examples 13-19, the risk of infection was evaluated to be high regardless of the number of liquid particles released. That is, similar evaluation results could be obtained in the range of the amount of liquid fine particles released from 50 to 517 mg/m³.

However, in Examples 20-22, the infection risk was evaluated to be low and showed a different tendency from Examples 13-19. This can be said to indicate that when the number of liquid particles released decreases, it becomes more susceptible to the influence of the number of particles present in the natural environment, and it may become impossible to perform accurate evaluations depending on the measurement conditions. That is, it can be said that it is preferable to emit enough liquid particles, specifically, 50 mg/m³ or more so that the influence of the number of particles existing in the natural environment can be ignored.

### [Improvement Effect of Air Purifiers and Fans on Infection Risk]

### Example 23

In the laboratory 1 shown in FIGS. 1 and 2, 300 mg/m³ of liquid particles 4 are discharged from the smoke machine 2, and while the air purifier and electric fan are running, the particle measuring device 6 measures the amount of liquid with a particle size of 0.3 μm. The decrease in the number of microparticles 4 was tracked.

Note that the ventilation volume of the ventilation fan 8 was set to 400 m³/hr. As the air purifier, a humidifying air purifier (trade name: KC-G40-W, manufactured by Sharp) was used and operated under conditions to purify 240 m³ of air per hour. As the electric fan, a living room electric fan (product name: AMT-KC30, manufactured by Yamazen) was used and was operated at maximum air volume. The evaluation results are shown in Table 5 and FIG. 6.

### Examples 24 and 25

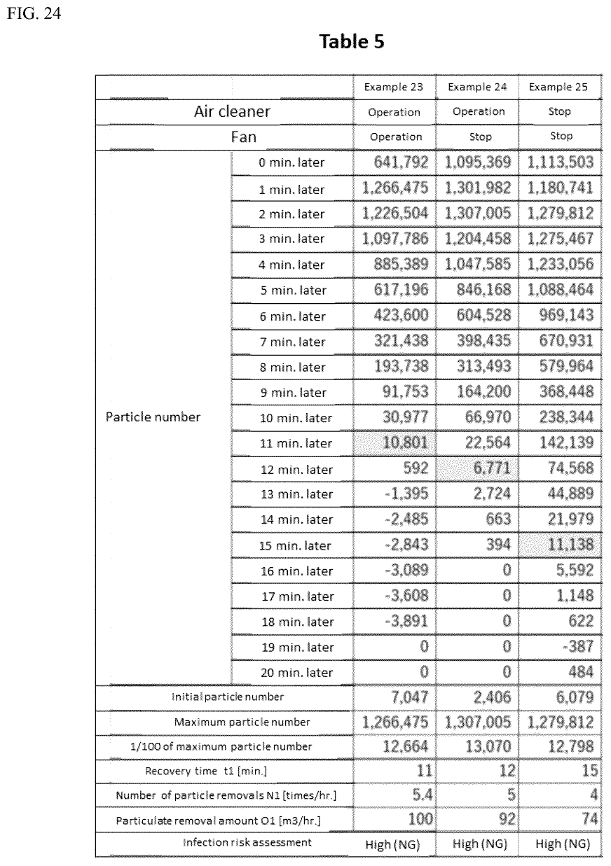

The decrease in the number of liquid particles was tracked in the same manner as in Example 23, except that the operating conditions of the air cleaner and electric fan were changed as shown in Table 5 of FIG. 24. The results are shown in Table 5 of FIG. 24 and FIG. 6.

The recovery time t1 is shorter in the order of Example 25, in which both the air cleaner and the electric fan were stopped, Example 24, in which only the air cleaner was operated, and Example 23, in which both the air cleaner and the electric fan were operated. In other words, operating an air purifier or electric fan was found to be effective in reducing the risk of infection. However, Examples 24 and 23 were not evaluated as having a “low risk of infection,” and it was recognized that additional improvement measures were required.

### [Improvement Effect of Air Conditioner on Infection Risk]

### Example 26

In the laboratory 1 shown in FIGS. 1 and 2, 300 mg/m³ of liquid particles 4 are discharged from the smoke machine 2, and the quantity of liquid particles 4 with a particle diameter of 0.3 μm is measured by the particle counter 6 while the air conditioner is running.

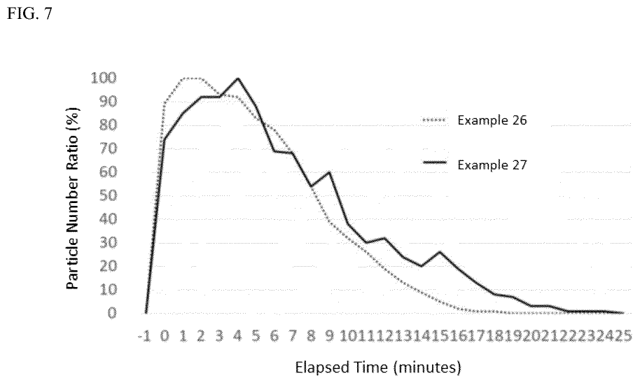

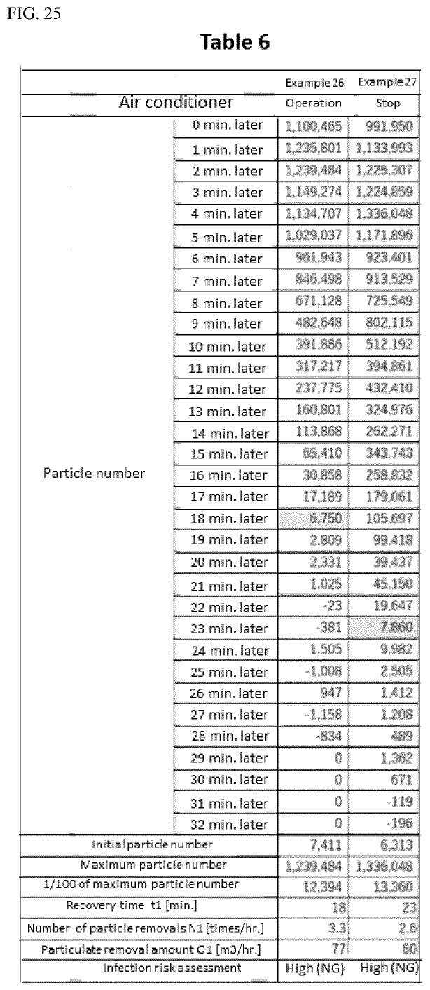

Note that the ventilation volume of the ventilation fan 8 was set to 400 m³/hr. The air conditioner was manufactured by Corona, trade name: CSH-B2220R, and was operated at maximum cooling/air volume. The evaluation results are shown in Table 6 of FIG. 25 and FIG. 7.

### Example 27

The decrease in the number of liquid particles was tracked in the same manner as in Example 26, except that the air conditioner was stopped. The results are shown in Table 6 of FIG. 25 and FIG. 7.

Compared to Example 27 in which the air conditioner was stopped, Example 26 in which the air conditioner was operated had a shorter recovery time t1. In other words, running an air conditioner was found to influence reducing the risk of infection. However, Example 26 was not evaluated as having a “low risk of infection,” and it was recognized that additional improvement measures were necessary.

### [Example 1 of Improving Infection Risk at Physical Stores]

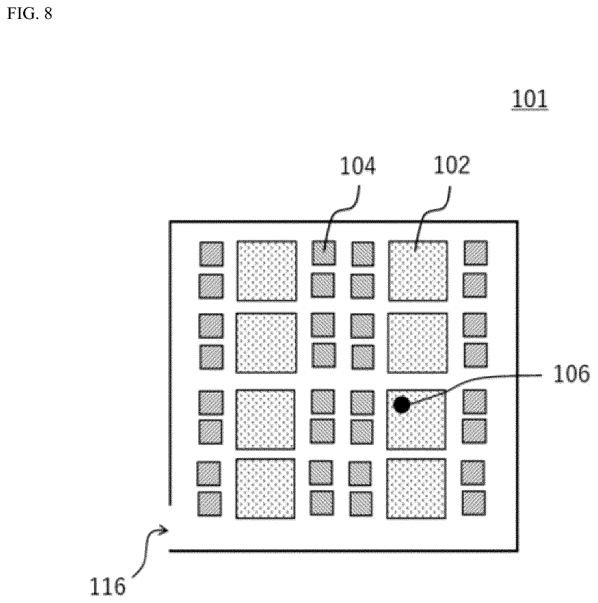

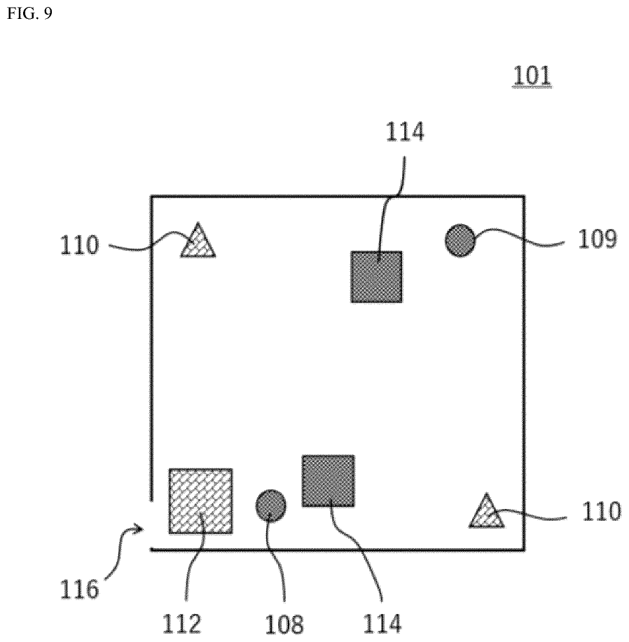

The evaluation of Examples 28-32 was conducted in the hall 101 of the pub shown in FIGS. 8 and 9. Hall 101 is a room measuring 4.9 m long (vertical direction in FIG. 8)×4.85 m wide (horizontal direction in FIG. 8)×2.69 m high, with a floor area of 23.8 m² and a volume V of 63.9 m³.

The capacity number n of hall 101 was 14 people, and the required ventilation amount per customer was 30 [m³/hr.]. From these values and equation (2), the human ventilation requirement Q2 [m³/hr.] is calculated as 420 [m³/hr.]. In addition, the hall 101 falls under the category of “cafeteria, restaurant, sushi restaurant” in the hypothetical sanitary laboratory guidelines, and the required number of ventilations N2 is 6 times. From this value and equation (4), the required ventilation time t2 [minutes] is calculated as 10 [minutes].

Furthermore, Hall 101 falls under the category of “Restaurant/Café” according to the Air Conditioning and Sanitary Engineers Society Standards (HASS 102 1972), and the required ventilation amount per floor area is 17.7 [m³/m2/hr.]. It is 23.8m2. From these values and equation (5), the area required ventilation Q3 [m³/hr.] is calculated as 420 [m³/hr.].

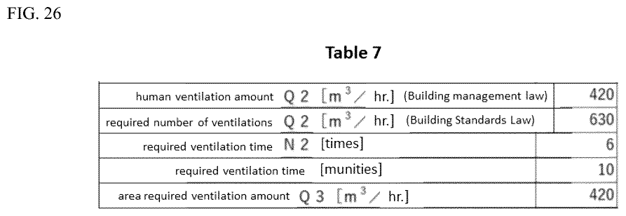

Table 7 of FIG. 26 shows the required human ventilation amount Q2, the required number of ventilations N2, the required ventilation time t2, and the required area ventilation amount Q3 in the laboratory 1 under the above conditions. Compare these values with the measured values to determine the relationship: particulate removal amount Q1≥required ventilation amount Q2 (or required area ventilation amount Q3), number of particulate removal times N1≥required number of ventilations N2, and recovery time t1≤required ventilation time t2. If the criteria are met, the risk of infection is low; if not, the risk of infection is assessed to be high.

### Example 28

In the hall 101 shown in FIGS. 8 and 9, 300 mg/m³ of liquid particles are discharged from the smoke machine, and the air purifiers 110 (2 units), the air conditioners 114 (2 units), and the total heat exchanger 112 are operated, the decrease in the number of liquid particles with a particle diameter of 0.3 μm was tracked using the particle counter 106.

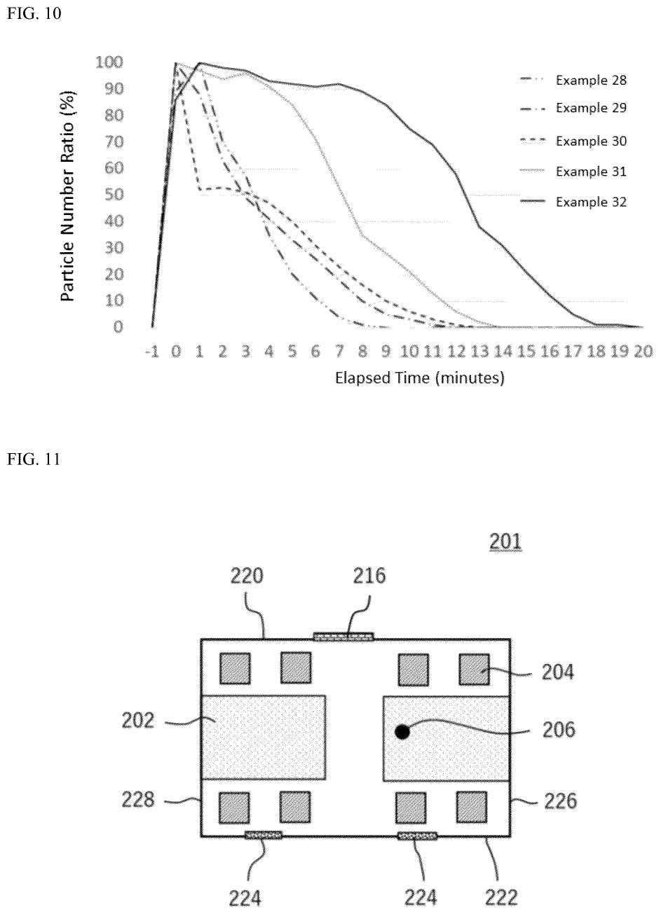

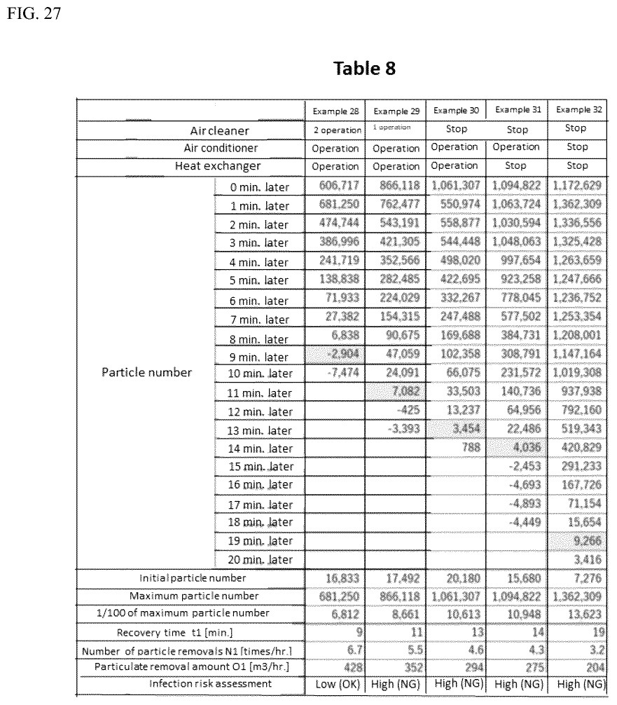

Note that the ventilation volume of the ventilation fans 108 (two units) was set to 170 m³/hr., and the ventilation volume of the ventilation fan 109 was set to 78 m³/hr. As the air cleaner 110, a humidifying streamer air cleaner (trade name: ACK55X, manufactured by Daikin Industries, Ltd.) was used and operated under conditions to purify 330 m³ of air per hour. As the air conditioner 114, a stationary commercial air conditioner (model unknown) was used, and was operated at maximum cooling and air volume. As the total heat exchanger 112, a total heat exchanger unit (trade name: VAH500 GB, manufactured by Daikin Industries) was used, and the ventilation rate was set at 500 m 3/hr. The evaluation results are shown in Table 8 of FIG. 27 and FIG. 10.

### Examples 29-32

The decrease in the number of liquid particles was tracked in the same manner as in Example 28, except that the operating conditions of the air cleaner, air conditioner, and total heat exchanger were changed as shown in Table 8 of FIG. 27. The results are shown in Table 8 of FIG. 27 and FIG. 10.

Example 32, in which the air purifier, air conditioner, and total heat exchanger were all stopped; Example 31, in which only the air conditioner was operated; Example 30, in which the air conditioner and the total heat exchanger were operated; one air purifier; and the air conditioner. The recovery time t1 becomes shorter in the order of Example 29, in which a total heat exchanger was operated, and Example 28, in which two air cleaners, an air conditioner, and a total heat exchanger were operated.

In other words, operating an air purifier, air conditioner, or total heat exchanger was found to be effective in reducing infection risk. Furthermore, regarding Example 28, these improvement measures made it possible to improve the hole 101 shown in FIGS. 8 and 9.

### [Example 2 of Improving Infection Risk at Physical Stores]

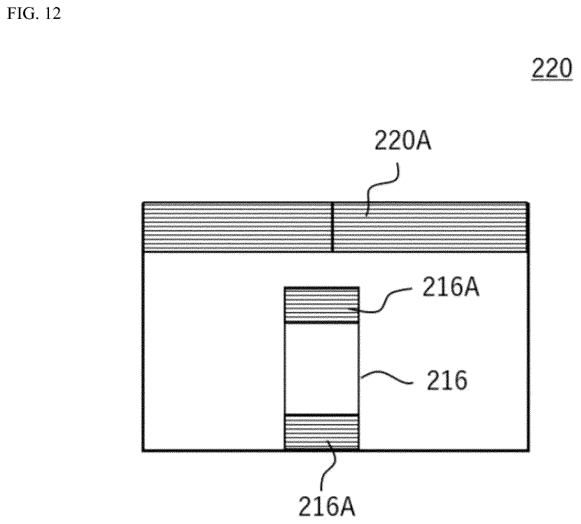

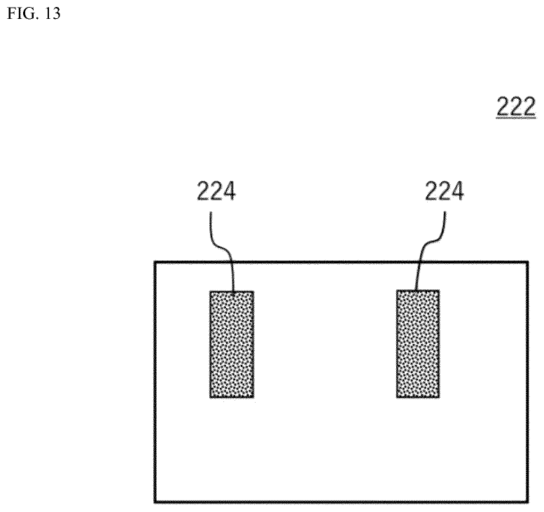

The evaluation of Examples 33-34 was conducted in a semi-private room 201 of a pub shown in FIG. 11. The semi-private room 201 is a room measuring 2.3 m long (vertical direction in FIG. 11)×3 m wide (horizontal direction in FIG. 11)×2.7 m high, with a floor area of 6.9 m² and a volume V of 18.6 m³. The semi-private room 201 is not equipped with an air conditioner or ventilation fan. Furthermore, as shown in FIG. 13, two windows 224 communicating with the outside are formed in one wall 222 of the four walls that partition the semi-private room 201, and as shown in FIGS. Transoms 220A, 226A, and 228A are formed at the top of the three walls 220, 226, and 228. Furthermore, as shown in FIG. 12, crosspieces 216A are incorporated in the upper and lower portions of the sliding door 216 to form a plurality of gaps. The transom part of the wall and the crosspiece part of the door communicate with the outside of the semi-private room, and have a structure that allows ventilation.

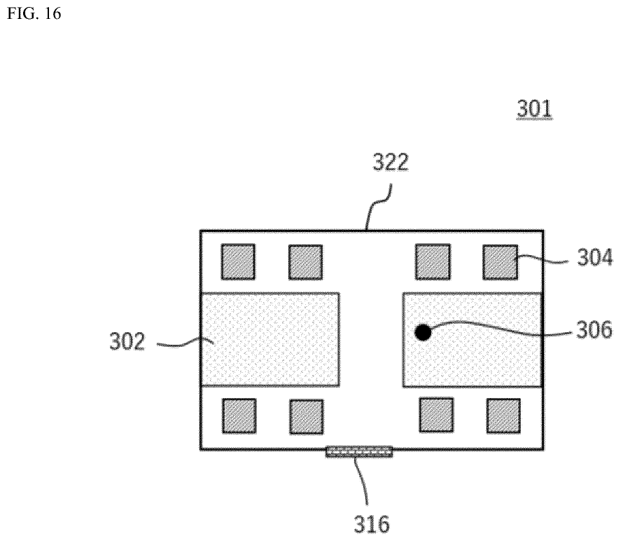

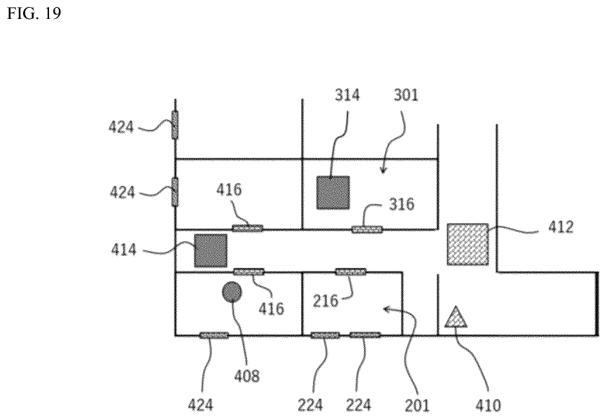

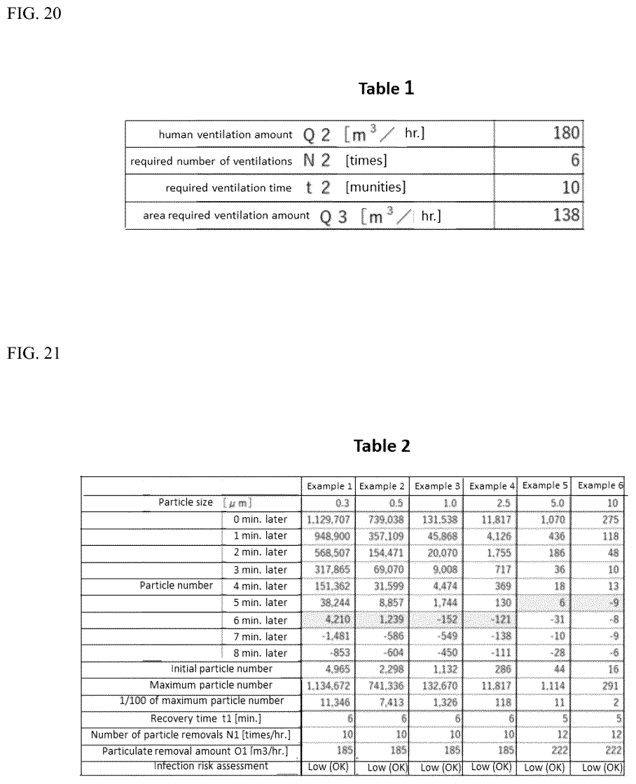

FIG. 19 shows air conditioning equipment for a store including the semi-private room 201 of the pub shown in FIG. 11 and the semi-private room 301 shown in FIG. 16, which will be described later. A ventilation fan 408, an air cleaner 410, a total heat exchanger 412, an air conditioner 414, a sliding door 416, and a window 424 are installed outside the semi-private room 201 and the semi-private room 301. In Tables 8 and 10, the “open” window refers to a state where not only the window 224 but also the window 424 is open, and the “closed” window refers to a state where the window 224 and the window 424 are closed. Furthermore, “running” the air conditioner refers to a state in which not only the air conditioner 314 but also the air conditioner 414 is operating, and “stopping” the air conditioner refers to stopping the air conditioner 314 and the air conditioner 414. Further, “running” and “stopping” of the air cleaner refer to running and stopping the air cleaner 410, and “running” and “stopping” of the total heat exchanger refer to running and stopping the total heat exchanger 412.

The capacity of the semi-private room 201 was 8 people, and the required ventilation amount per customer was 30 [m³/hr.]. From these values and equation (2), the required human ventilation Q2 [m 3/hr.] is calculated as 240 [m³/hr.]. In addition, the semi-private room 201 falls under the category of “cafeteria, restaurant, sushi restaurant” in the hypothetical sanitary laboratory guidelines, and the required number of ventilations N2 is 6 times. From this value and equation (4), the required ventilation time t2 [minutes] is calculated as 10 [minutes].

Furthermore, the semi-private room 201 falls under the category of “restaurant/cafe” according to the Air Conditioning and Sanitary Engineers Society Standards (HASS 102 1972), and the required ventilation per floor area is 30 [m³/m²/hr.], and the floor area is 6. It is 9 m². From these values and equation (5), the area required ventilation Q3 [m³/hr.] is calculated as 207 [m³/hr.].

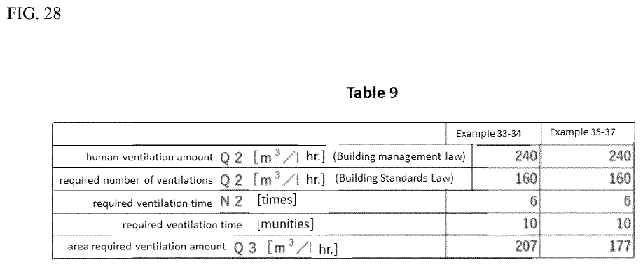

Table 9 in FIG. 28 shows the required human ventilation rate Q2, required ventilation frequency N2, required ventilation time t2, and area required ventilation rate Q3 for the semi-private room 201 under the above conditions. Compare these values with the measured values to find the relationship: particulate removal amount Q1≥required ventilation amount Q2 (or required area ventilation amount Q3), number of particulate removal times N1≥required number of ventilations N2, and recovery time t1≤required ventilation time t2. If the criteria are met, the risk of infection is low; if not, the risk of infection is assessed to be high.

### Example 33

In the semi-private room 201 shown in FIGS. 11 and 19, 300 mg/m³ of liquid particles are emitted from the smoke machine, the windows 224 and 424 are closed, and the ventilation fan 408, air purifier 410, and total heat exchanger 412 are operated, the decrease in the number of liquid particles with a particle size of 0.3 μm was tracked using the particle counter 206.

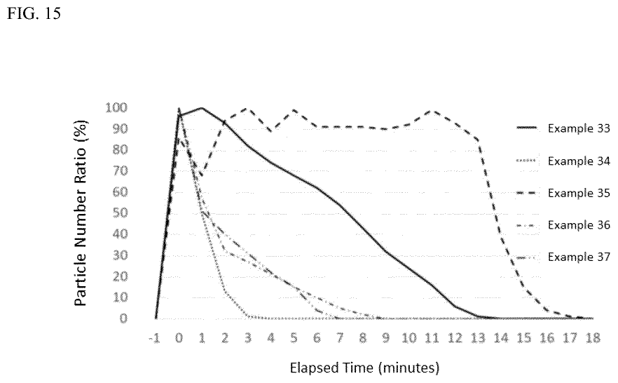

Note that the air cleaner 410 and total heat exchanger 412 were of the same model as those used in Example 28, and were operated under the same operating conditions as Example 28. The evaluation results are shown in Table 10 and FIG. 15.

### Example 34

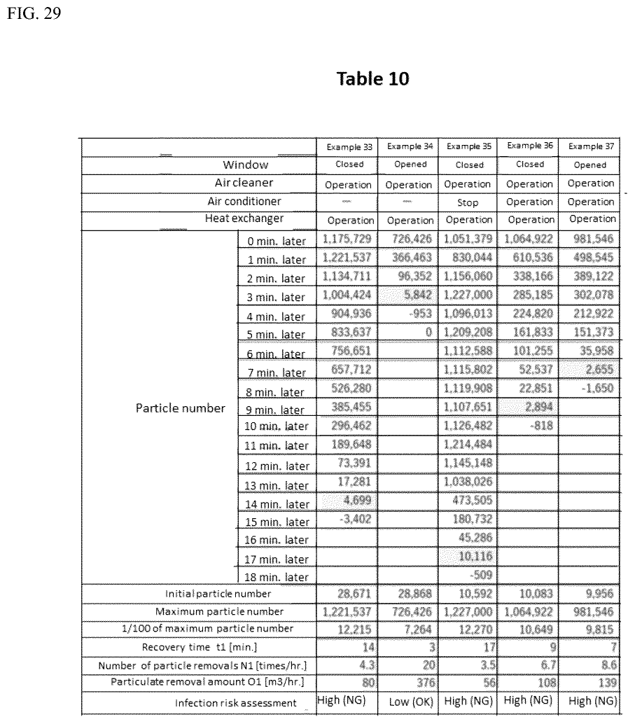

The quantity of liquid particles was the same as in Example 33, except that the opening/closing status of the windows 224 and 424 and the operating status of the air cleaner 410 and total heat exchanger 412 were changed as shown in Table 10 of FIG. 29. tracked the decline in the results are shown in Table 10 of FIG. 29 and FIG. 15.

From Example 33 in which the windows 224 and 424 were closed and the air cleaner 410 and the total heat exchanger 412 were operated, the windows 224 and 424 were opened and the air cleaner 410 and the total heat exchanger 412 were operated. The recovery time t1 in Example 34 is significantly shorter. That is, it was recognized that opening the windows 224 and 424 had an effect of improving the risk of infection. In addition, in Example 34, these improvement measures made it possible to improve the semi-private room 201 shown in FIGS. 5 and 6 to the level of “low risk of infection”, and a particularly good improvement effect was obtained.

### [Example 3 of Improving Infection Risk at Physical Stores]

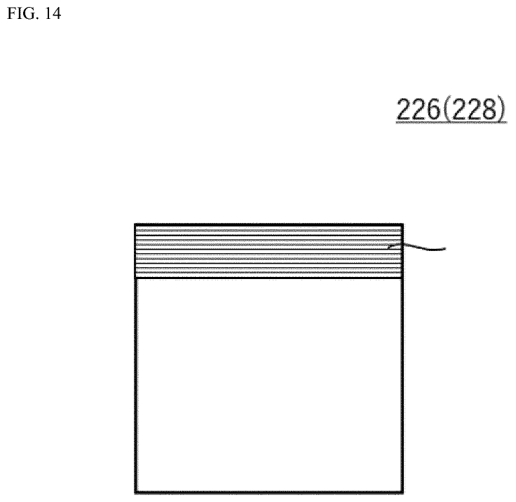

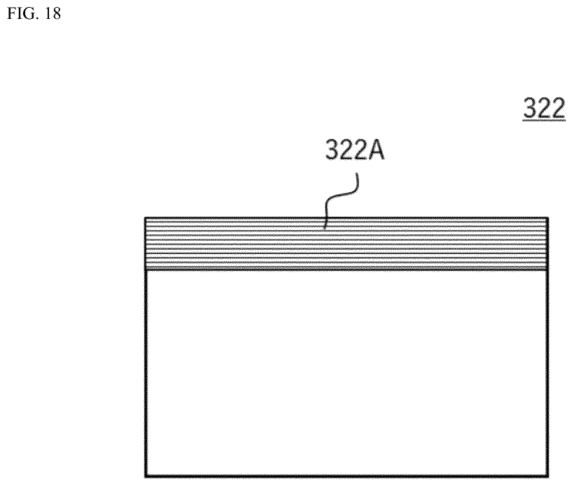

The evaluation of Examples 35-37 was conducted in a semi-private room 301 of a pub shown in FIGS. 16 and 19. The semi-private room 301 is 1.8 m long (vertical direction in FIG. 16)×3.3 m wide (horizontal direction in FIG. 16)×2.7 m high, has a floor area of 5.9 m², and a volume V of 16 m³. The semi-private room 301 has the same structure as the semi-private room 201 shown in FIG. 11, except that it does not have a window. Specifically, the wall with sliding door 316 is configured like wall 220 shown in FIG. 12, and the two walls adjacent to the wall with sliding door 316 are configured like walls 226, 228 shown in FIG. 14. As shown in FIG. 18, the wall 322 facing the wall having the sliding door 316 has a transom 322A formed in the upper part of the wall 322. That is, transoms are formed at the top of the four walls that partition the semi-private room 301, and bars are built into the top and bottom of the sliding door 316 to form a plurality of gaps. The transom part of the wall and the crosspiece part of the door communicate with the outside of the semi-private room, and have a structure that allows ventilation.

The capacity of the semi-private room 301 was 8 people, and the required ventilation amount per customer was 30 [m³/hr.]. From these values and equation (2), the required human ventilation Q2 [m 3/hr.] is calculated as 240 [m³/hr.]. In addition, the semi-private room 301 falls under the category of “cafeteria, restaurant, sushi restaurant” in the hypothetical sanitary laboratory guidelines, and the required number of ventilations N2 is 6 times. From this value and equation (4), the required ventilation time t2 [minutes] is calculated as 10 [minutes].

Furthermore, the semi-private room 301 falls under the category of “restaurant/café” according to the Air Conditioning and Sanitary Engineers Society Standards (HASS 102 1972), and the required ventilation amount per floor area is 30 [m³/m2/hr.], and the floor area is 5. It is 9 m². From these values and equation (5), the area required ventilation Q3 [m³/hr.] is calculated as 177 [m³/hr.].

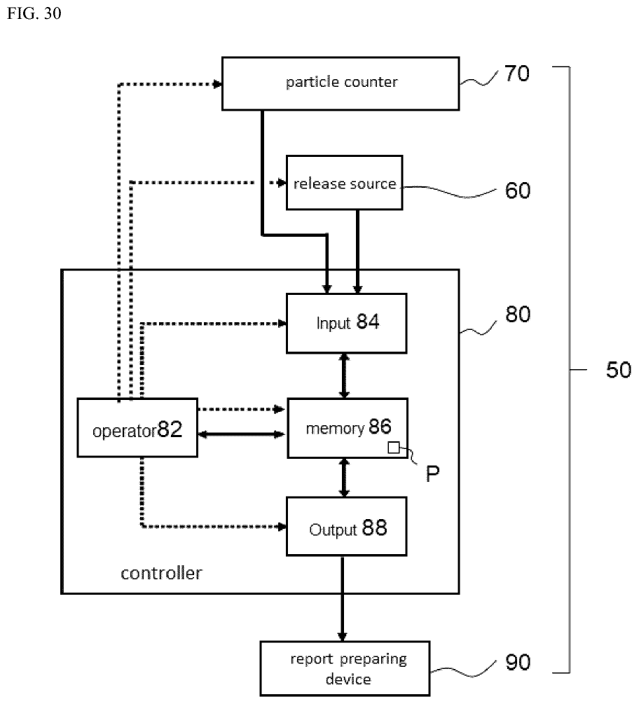

Table 11 in FIG. 30 shows the required human ventilation rate Q2, required ventilation frequency N2, required ventilation time t2, and area required ventilation rate Q3 for the semi-private room 301 under the above conditions. Compare these values with the measured values to find the relationship: particulate removal amount Q1≥required ventilation amount Q2 (or required area ventilation amount Q3), number of particulate removal times N1≥required number of ventilations N2, and recovery time t1≤required ventilation time t2. If the criteria are met, the risk of infection is low; if not, the risk of infection is assessed to be high.

### Example 35

In the semi-private room 301 shown in FIGS. 16 and 19, 300 g/m³ of liquid particles are discharged from the smoke machine, the windows 224, 424 are closed, the ventilation fan 408, the air purifier 410, and the total heat exchanger 412 are operated, and the air conditioners 314, 414 was stopped, the particle counter 306 tracked the decrease in the number of liquid particles with a particle diameter of 0.3 μm.

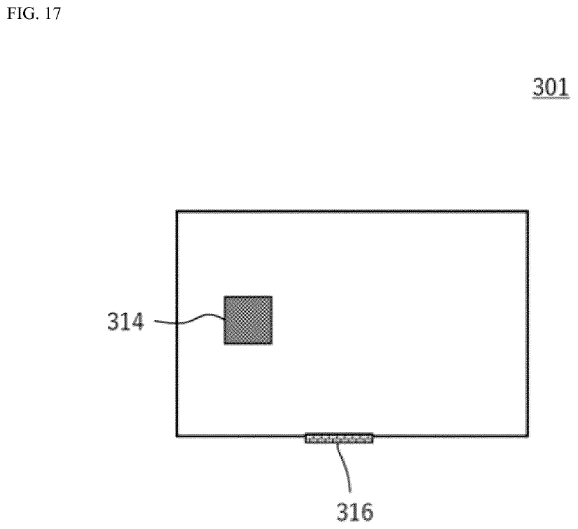

FIGS. 17 and 19 show the arrangement of the air conditioner 314 and the total heat exchanger 412. Note that the air cleaner 410 and total heat exchanger 412 were of the same type as those used in Example 28, and were operated under the same operating conditions as Example 28. As the air conditioners 314 and 414, ceiling-embedded air conditioners (product name: FHCP160EC, manufactured by Daikin Industries) were used and operated at maximum cooling and air volume. The evaluation results are shown in Table 10 of FIG. 29 and FIG. 15.

### Examples 36 and 37

The decrease in the number of liquid particles was tracked in the same manner as in Example 35, except that the operating conditions of the air cleaner 410, air conditioners 314, 414, and total heat exchanger 412 were changed as shown in Table 10. The results are shown in Table 10 of FIG. 29 and FIG. 15.

Example 35, in which the windows 224, 424 were closed, the air conditioners 314, 414 were stopped, and the air purifier 410 and the total heat exchanger 412 were operated; Example 36, in which the 414 and total heat exchanger 412 were operated, and Example 37, in which the windows 224, 424 were opened and all of the air cleaner 410, the air conditioners 314, 414, and the total heat exchanger 412 were operated. The recovery time t1 is shorter. In other words, it was found that opening windows and operating air purifiers, air conditioners, and total heat exchangers reduced the risk of infection. However, none of Examples 35 to 37 were evaluated as having a “low risk of infection,” and it was recognized that additional improvement measures were necessary.

Note that even under the same conditions as in Example 37, the semi-private room 301 can be made into a “low infection risk” state by additionally installing an air purifier or total heat exchanger, or by reducing the capacity of the semi-private room 301. It is possible to improve the evaluation to “low”.

For example, in Example 37, the particulate removal amount Q1 is 139 [m³/hr.], which is lower than the required human ventilation rate Q2 of 240 [m³/hr.], so the risk of infection is evaluated to be high. However, if the capacity n of the semi-private room 301 is reduced from 8 to 4, the required human ventilation Q2 will drop to 120 [m³/hr.]. In other words, if the accommodating capacity n of the semi-private room 301 is 4 people, the amount of particulate removal Q1 (139 [m³/hr.]) exceeds the required human ventilation rate Q2 of 120 [m³/hr.], and the risk of infection is evaluated to be low.

### «Summary and Supplement for 4.1-3»

Hereinafter, the summary and supplements of 1 to 3 created in the first period will be explained.

### SUMMARY

The descriptions in 1 to 3 and the drawings referred to include the following inventions. Here, a plurality of inventions are described as appendices 1 to 13, respectively.

### [Appendix 1]

An infection risk assessment method for assessing the risk of infection in an indoor gathering space of a facility, including:
the risk of infection is the risk of aerosol infection caused by aerosols containing pathogens, releasing liquid particles into the gathering space,
after discharging the liquid particles, tracking the decrease in the amount of the liquid particles over time,
based on the recovery time t1, which is the time required for the number of liquid particles to sufficiently decrease,
evaluating the particulate removal ability of the gathering space;
the particulate removal capacity is compared with the required ventilation capacity of the gathering space, and if the particulate removal capacity is equal to or higher than the required ventilation capacity, the risk of aerosol infection in the gathering space is evaluated to be low.

### [Appendix 2]

The infection risk assessment method described in Appendix 1, among the liquid particles, a decrease in the number of liquid particles with a particle diameter of 0.3 μm or more and 10 μm or less is tracked over time.

### [Appendix 3]

The infection risk assessment method described in Appendix 1, a device that tracks the decrease in the number of liquid particles using a particle counter.

### [Appendix 4]

The infection risk assessment method described in Appendix 1, evaluate the particulate removal ability by the particulate removal amount Q1 calculated by the following formula (1),
the required ventilation amount is defined by the required human ventilation amount Q2 calculated by the following formula (2),
the particulate removal amount Q1 is compared with the required human ventilation amount Q2, and if the particulate removal amount Q1 is equal to or higher than the required human ventilation amount Q2, the risk of aerosol infection in the gathering space is determined to be low. What to evaluate.

Particulate removal amount Q1 [m³/hr.]=Volume of gathering space V [m³]×60 [minutes]/Recovery time t1 [minutes] formula (1)

Required ventilation amount for each person Q2 [m³/hr.]=Required ventilation amount per customer [m³/hr./person]×Number of people in the gathering space n [persons] formula (2)

### [Appendix 5]

The infection risk assessment method described in Appendix 1, evaluate the particulate removal ability by the number of particulate removal times N1 or recovery time t1 calculated by the following formula (3),
the required ventilation amount is defined by the required ventilation number N2 of the gathering space determined in accordance with the Hygiene Laboratory guidelines or the required ventilation time t2 calculated by the following formula (4),
when comparing the number of particulate removal times N1 and the required number of ventilations N2, or the recovery time t1 and the required ventilation time t2, the number of particulate removals N1 is equal to or greater than the number of required ventilations N2, or When the recovery time t1 is equal to or greater than the required ventilation time t2, the risk of aerosol infection in the gathering space is evaluated to be low.

Number of particulate removals N1 [times/hr.]=60 [minutes]/Recovery time t1 [minutes] formula (3)

Required ventilation time t2 [minutes]=60 [minutes]/Required number of ventilations N2 [times/hr.] formula (4)

### [Appendix 6]

The infection risk assessment method described in Appendix 1, evaluate the particulate removal ability by the particulate removal amount Q1 calculated by the following formula (1),
the required ventilation amount is defined by the area required ventilation amount Q3 calculated by the following formula (5),
when the particulate removal amount Q1 is compared with the area required ventilation amount Q3, and the particulate removal amount Q1 is equal to or greater than the area required ventilation amount Q3, the aerosol infection risk in the gathering space is determined to be low. What to evaluate.

$$Particulate removal amount Q 1 [ {m}^{3} / hr . ] = Volume of gathering space V [ {m}^{3} ] \times 60 [ minutes ] / Recovery time t 1 [ minutes ] \qquad formula ( 1 )$$

$$Area required ventilation amount Q 3 [ {m}^{3} / hr . ] = Required ventilation amount per floor area [ {m}^{3} / {m}^{2} / hr . ] \times Floor area of gathering space [ {m}^{2} ] \qquad Formula ( 5 )$$

(However, the amount of ventilation required per floor area shall be the value specified in the Air Conditioning and Sanitary Engineers Society Standards (HASS 102 1972).)

### [Appendix 7]

The infection risk assessment method described in Appendix 1, one that releases liquid particles containing ethylene glycol and water into the gathering space.

### [Appendix 8]

The infection risk assessment method described in Appendix 1, a device that releases at least 50 mg/m³ of the liquid particles into the gathering space.

### [Appendix 9]

The infection risk assessment method described in Appendix 1, after discharging the liquid particles into the collection space, the liquid particles are reduced to 1/100 of the maximum number of particles within the shorter of 60 minutes or 60 minutes, whichever is shorter. Tracks the number of liquid particles over time.

### [Appendix 10]

The infection risk assessment method described in Appendix 1,
the risk of infection is the risk of aerosol infection caused by aerosols containing the new coronavirus, this evaluates the risk of aerosol infection of the new coronavirus infection (COVID-19) in the gathering space.

### [Appendix 11]

The infection risk assessment method described in Appendix 1,
the gathering space is not a space exclusively used by employees, but is a customer-use space that can be accessed by customers or customers and employees.

### [Appendix 12]

An infection risk reduction method that reduces the risk of infection in an indoor gathering space of a facility,
evaluate the aerosol infection risk in the gathering space using the infection risk assessment method described in Appendixes 1 to 11,
if the risk of aerosol infection in the gathering space is assessed as high, improvement measures will be taken until the particulate removal capacity is equal to or higher than the required ventilation capacity.

### [Appendix 13]

The infection risk reduction method described in Appendix 12,
the improvement measures include ventilation by opening windows, doors, or vents; operation of ventilation fans, total heat exchangers, air conditioners, circulators, or air purifiers;
installation of shielding plates or bulkheads;
reduction of accommodation capacity; and,
at least one measure selected from the group consisting of.

### <Supplement>

The liquid particles in Supplementary Notes 1 to 13 may be particles other than liquid. Alternatively, it may be a mixture of liquid particles and solid particles. That is, the liquid particles in Supplementary Notes 1 to 13 may be replaced with solid particles, or the term fine particles may be used simply as a general expression.

### «5. Evaluating Apparatus and Program for Aerosol Infection Risk»

Next, the evaluating apparatus 50 for an aerosol infection risk (hereinafter simply referred to as the evaluating apparatus 50) of the present embodiment will be described in order of the structure, function, operation, and effect of the evaluating apparatus 50, with reference to the drawings.

### <Structure and Function of the Evaluating Apparatus of the Present Embodiment>

FIG. 30 is a block diagram of the evaluating apparatus of this embodiment. The evaluating apparatus 50 includes the release source 60 (an example of a release device), the particle counter 70 (an example of an identifying device), the control device 80 (an example of a controller and an evaluating device), and the report preparing device 90 (an example of a preparing device).

### [Release Source and Particle Counter]

The release source 60 is arranged in a space having a defined volume (as an example, the above-mentioned gathering space) and has the function of releasing a plurality of fine particles into this space. The release source 60 may be, as an example, a machine that releases a plurality of particles, such as a commercially available smoke machine. Further, the type of particles released from the release source 60 may be liquid particles, solid particles, or a mixture thereof.

The particle counter 70 has functions of measuring the number of multiple fine particles multiple times in this space; deriving a specific elapsed time until the number of particles decreases to a predetermined number based on the relationship between the elapsed time from when the release source 60 finished releasing the plurality of particles and the number of the plurality of particles to be measured; and specifying the ability to reduce a plurality of particles within the space from the specific elapsed time.

Here, the “predetermined number” means “the number that is 1/100 the number measured as the maximum number of the plurality of liquid particles present in the air in the gathering space after the releasing step”. Along with this, the “specific elapsed time” means the time corresponding to the recovery time t1.

### [Control Device]

As shown in FIG. 30, the control device 80 includes a calculation section 82 (another example of a control section, an example of an evaluation section), an input section 84, a storage section 86, and an output section 88.

The input unit 84 has a function of receiving input signals from an external device. In this embodiment, as an example, the emission source 60 and the particle counter 70 correspond to external devices. The output section 88 has a function of outputting to the outside the result of the arithmetic processing performed by the arithmetic section 82 on the data input to the control device 80. In this embodiment, as an example, the report creation unit 90 corresponds to an external device, and the result is obtained by applying arithmetic processing to data input to the input unit 84 by the calculation unit 82 using a program P described later. Corresponds to the output data.

The calculation unit 82 has a function of performing calculation processing based on a program P (aerosol infection risk evaluation program) to be described later, and components other than the calculation unit 82 in the control device 80, the emission source 60, the particle counter 70, and the report creation unit. It has the function of controlling 90.

The storage unit 86 has a function of storing the program P and the results calculated by the calculation unit 82.

Note that the algorithm of the program P and the content of the arithmetic processing of the arithmetic unit 82 will be explained in the explanation of the operation of the evaluation device 50, which will be described later.

### [Report Creation Department]

The report creation unit 90 has a function of creating a report from the evaluation results evaluated by the control device 80 and information accompanying the evaluation results. The contents of the report will be explained later in the explanation of the operation of the evaluation device 50.

Although the evaluation device 50 of the present embodiment is provided with the report creation section 90 as an example, as will be described later, it is possible to realize the evaluation of aerosol infection risk even without the report creation section 90. From this point of view, the report creation section 90 may be omitted.

<Operation of the Evaluation Device of this Embodiment (Aerosol Infection Risk Evaluation Method)>

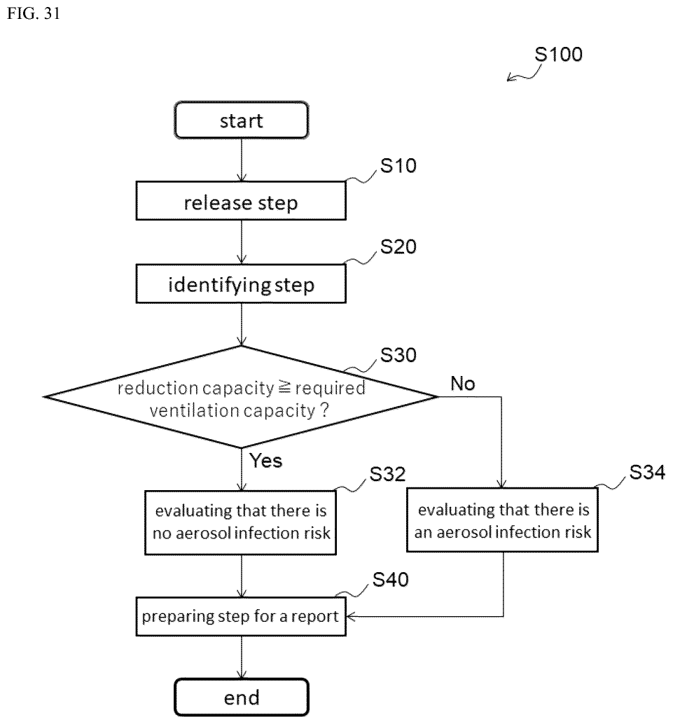

Next, the operation of the evaluation device 50 of this embodiment (aerosol infection risk evaluation method S100 of this embodiment) will be described with reference to FIGS. 30 and 31.

FIG. 31 is a flowchart of an aerosol infection risk evaluation method performed using the evaluation device 50 of this embodiment. The aerosol infection risk evaluation method of this embodiment includes the following steps.
Release process S10
Specific process S20
Step S30 of determining whether the reduction capacity is greater than or equal to the required ventilation capacity (determination step S30)
Evaluation step S32 to evaluate that there is no risk of infection (affirmative evaluation step S32)
Evaluation step S34 of evaluating that there is a risk of infection (negative evaluation step S34)
Report creation process S40

The contents of each of the above steps will be described below. In each step, the calculation section 82 that performs calculation processing based on the program P stored in the storage section 86 controls the components other than the calculation section 82 in the evaluation device 50. This is achieved by

### [Release Step S10]

The aerosol infection risk evaluation method of this embodiment starts from the release step S10. The release step S10 is a step in which the release source 60 placed in a space having a predetermined volume releases a plurality of fine particles into the space. Specifically, the calculation unit 82 of the control device 80 operates the emission source 60, so that the emission source 60 placed in the space to be evaluated releases a plurality of particles into the space for a predetermined period of time. This process ends when a state in which a plurality of fine particles are included in the space is formed. Note that the quantity of the plurality of particles released into the space is the same as in the plurality of embodiments described above.

### [Specific Process S20]

The specific step S20 is a step performed after the release step S10 is completed. The identification step S20 refers to measuring the quantity of a plurality of particles multiple times in the space from which the plurality of particles was released in the emission step S10, and determining the elapsed time from the end of the emission of the plurality of particles by the emission source 60. Deriving a specific elapsed time until the number of the plurality of particles decreases to a predetermined amount from the relationship with the number of the plurality of particles to be measured, and calculating the number of particles in the space from the specific elapsed time. This is the process of identifying the reduction ability of This step is performed by the particle counter 70 controlled by the calculation unit 82. Here, the predetermined quantity is, for example, a quantity that is 1/100 of the quantity measured as the maximum value of the quantity of a plurality of liquid particles present in the air in the space after the discharge step S10. In addition, the specific elapsed time is, for example, the period until the number measured as the maximum number of the plurality of liquid particles present in the air in the space after the discharge step S10 decreases to 1/100 of the number. This corresponds to the elapsed time (that is, the recovery time t1 described above). The ability to reduce a plurality of particles in a space is a value specified from a specific elapsed time. The longer the recovery time t1, the lower the reduction ability.

After the particle counter 70 specifies the reduction ability, the data on the reduction ability is stored in the storage unit 86 via the input unit 84, and this step ends.

### [Judgment Step S30, Positive Evaluation Step S32, and Negative Evaluation Step S34]

The determination step S30 is a step performed after the identification step S20, and is a step of determining whether the reduction capacity is greater than or equal to the required ventilation capacity. The positive evaluation step S32 and the negative evaluation step S34 are steps performed after the determination step S30, respectively. In the affirmative evaluation step S32, if an affirmative determination is made in the determination step S30, that is, if it is determined that the reduction capacity is greater than or equal to the required ventilation capacity, an affirmative evaluation (no (or low) risk of aerosol infection) is performed. This is the process of On the other hand, in the negative evaluation step S34, if a negative determination is made in the determination step S30, that is, if it is determined that the reduction capacity is less than the required ventilation capacity, a negative evaluation (there is a risk of aerosol infection (or This is a process that is evaluated as high)).

This process ends when the calculation unit 82 compares the reduction capacity data and the required ventilation capacity data stored in the storage unit 86 to determine whether the evaluation is positive or negative.

### [Report Creation Process S40]

The report creation step S40 is a step performed after the positive evaluation step S32 or the negative evaluation step S34. In the report creation step S40, the report creation section 90 creates a report from the evaluation results evaluated by the control device 80 (calculation section 82) and information accompanying the evaluation results. Here, the report containing information accompanying the evaluation results includes, as an example, any of the following contents.
Reduction ability specified in specific process S20
Information regarding the relationship between the elapsed time from the end of the emission of a plurality of particles by the emission source 60 and the quantity of the plurality of particles measured in the specific step S20 (for example, a table, a graph (2) dimensional spectrum) etc.)
If a negative judgment is made in the judgment step S30, information regarding the insufficient reduction capacity required to evaluate that there is no risk of aerosol infection in the space to be evaluated.
If a positive judgment is made in the judgment step S30, information regarding the extra reduction capacity required to evaluate that there is no risk of aerosol infection within the space to be evaluated.

The report creation unit 90 is, for example, a display device such as a display, a printing device such as a printer, a transmitting device that transmits a report to an information terminal used by a user via a network, or other devices.

This step ends when the report creation unit 90 creates the report. Furthermore, the aerosol infection risk evaluation method S100 of this embodiment ends at the end of the report creation process.

### [Program P Algorithm]

The algorithm of program P is an algorithm for realizing the operation based on the flowchart of FIG. 31, which is an example for executing the aerosol infection risk evaluation method S100 of this embodiment.

<Effects of the Evaluation Device of this Embodiment>

Next, the effects of the evaluation device 50, evaluation method, and program P of this embodiment will be described.

The aerosol infection risk evaluation method S100 of this embodiment easily evaluates whether there is an aerosol infection risk within a space having a defined volume by specifying the reduction ability obtained from the reduction rate of a plurality of fine particles.

Furthermore, the aerosol infection risk evaluation method S100 of the present embodiment can notify the user of the reduction ability through a report created using the report creation unit 90.

Furthermore, the aerosol infection risk evaluation method S100 of the present embodiment visualizes the relationship between the elapsed time from the end of the process of releasing a plurality of microparticles and the measured quantity of the plurality of microparticles, using a report that is created. You can let the user know.

Furthermore, in the aerosol infection risk evaluation method S100 of the present embodiment, when it is evaluated that there is an aerosol infection risk in a space, the report creation function of the report creation unit 90 evaluates the insufficient reduction ability in the space. Users can be informed. In addition, in the aerosol infection risk evaluation method S100 of the present embodiment, when it is evaluated that there is no aerosol infection risk in a space, the report creation function of the report creation unit 90 calculates the extra capacity of the reduction capacity in the space. Users can be informed.

Furthermore, the aerosol infection risk evaluation program (program P) of the present embodiment allows a computer (e.g., the evaluation device 50) to easily evaluate whether there is an aerosol infection risk within a space having a predetermined volume, can be done.

### «6. HACCP Type Aerosol Infection Risk Evaluation Device and Evaluation Program»

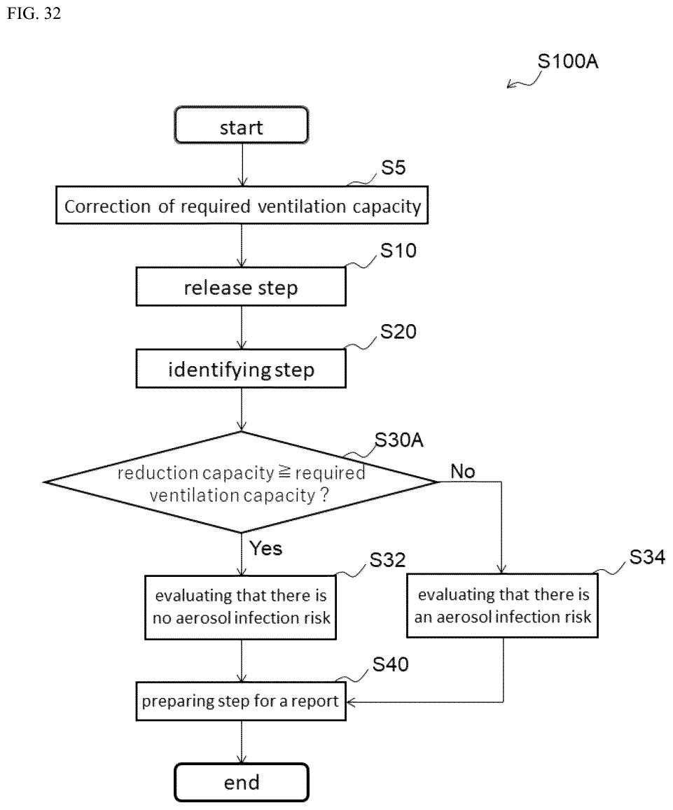

Next, a HACCP type aerosol infection risk evaluation device (hereinafter referred to as HACCP type evaluation device), which is a modified example of the aerosol infection risk evaluation device 50 of the present embodiment, will be described with reference to FIG. 32. The purpose of creating the HACCP type evaluation device, the configuration and functions of the HACCP type evaluation device, the operation, and the effects will be explained in this order. In the following description, only the points that are different from the evaluation device 50 described above will be described. Furthermore, in the following description, it should be noted that the same reference numerals are used to describe components having the same functions as the constituent elements of the present embodiment described above.

### <Purpose of Creating the HACCP Type Evaluation Device>

HACCP is an abbreviation for Hazard Analysis and Critical Control Point, and food business operators themselves must understand the hazards such as food poisoning bacterial contamination and foreign substance contamination, and then manage all processes from the receipt of raw materials to the shipment of products. It is a method of hygiene management that aims to ensure the safety of products by managing particularly important steps in the process to eliminate or reduce these hazardous factors. In other words, HACCP is a hygiene management method in the field of food and the like.

The required ventilation capacity explained so far is based on standards established simply by considering the volume of the space, but originally, depending on how the space is used, etc., the required ventilation capacity may vary even in a space with the same volume. It is considered that it is okay to be different. For example, even if a concert is held with 1,000 people gathered in the same volume of gathering space, the number of people who gather in the same volume will differ between a hard rock concert and a classical music concert. It is clear that there can be large differences in the amount of droplets created in a space.

Therefore, Tetsuya Mizuno, the inventor of this application, believes that incorporating the HACCP concept into hygiene management in the environmental field will become a true environmental hygiene management method, and has developed the HACCP type evaluation device and HACCP type described below. Created an evaluation program.

### <Function, Configuration, and Operation of HACCP Type Evaluation Device>

In this modification, the differences from the above-described embodiment are as follows. In other words, the evaluation flow in FIG. 32 (HACCP type aerosol infection risk evaluation method S100A) is based on the usage pattern of one or more users in the space to be evaluated. (see S5) is replaced with judgment step S30 (see judgment step S30A) of the evaluation flow described above (see FIG. 31).

As a result, for example, if the required ventilation capacity to be evaluated has a value of Q, for example, when the gathering space is a hard rock concert, etc., the value will be 5×Q based on past evaluation results, etc. During a concert, the value may be 0.3×Q based on past measurement results. Here, the respective “5×” and “0.3×” parts are weighting coefficients that are determined depending on how the gathering space is used. It may be stored in the storage unit 86. The weighting coefficient also depends on the mask usage status of the users using the gathering space, the speaking intensity of each user (how loud they speak), and the like. Therefore, these may be varied depending on the measured results.

### <Effects of HACCP Type Evaluation Equipment, Etc.>

According to this modification, it is possible to evaluate the risk of aerosol infection more appropriately.

### «7. Air Purification System»

Next, an air purification system 55 that includes the evaluation device 50 of the present embodiment described above (however, the report creation unit 90 may not be included) will be described. That is, multiple types of air cleaning systems will be described here.

<Air Cleaning System of this Embodiment>

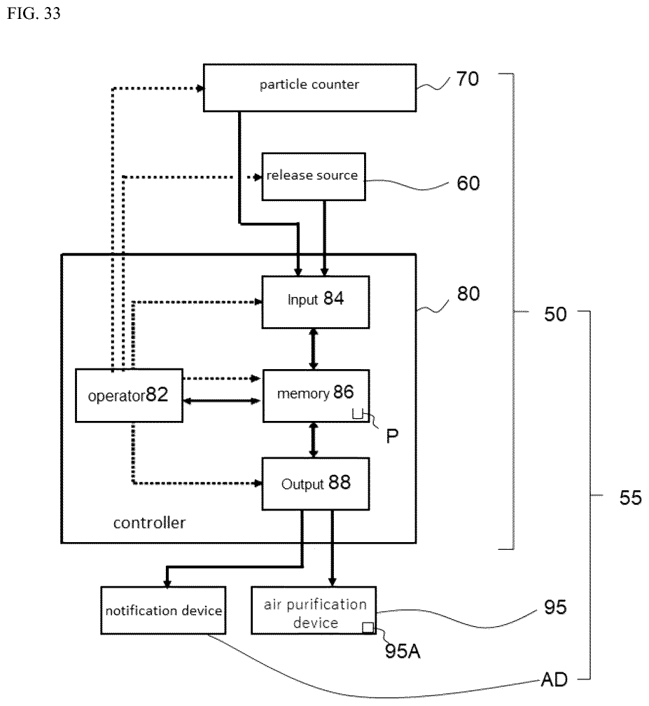

Hereinafter, the air cleaning system 55 of this embodiment will be explained with reference to FIGS. 33 to 35.

[Function, Configuration, and Operation of the Air Cleaning System of this Embodiment]

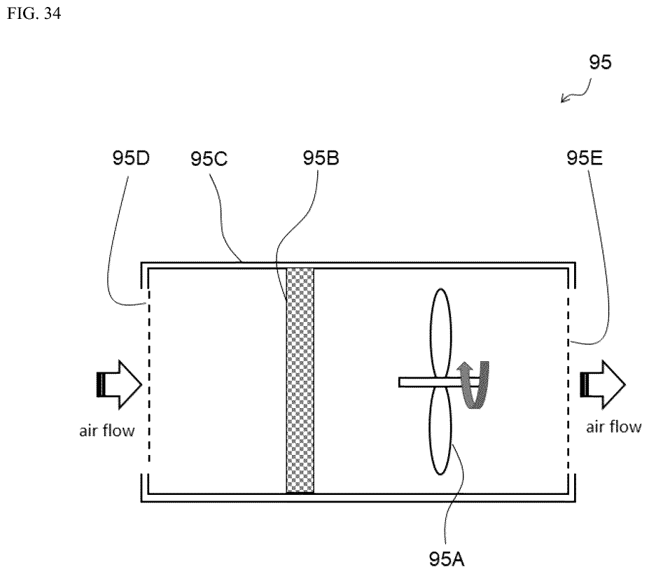

FIG. 33 is a block diagram of the air cleaning system 55 of this embodiment. Moreover, FIG. 34 is a schematic diagram of the air cleaning device 95 of this embodiment. The air purification system 55 of this embodiment includes the evaluation device 50 of this embodiment (however, the report creation section 90 may not be provided), the air purification device 95, and the notification device AD (an example of the notification section). The air purifying device 95 and the notification device AD are communicably connected to the evaluation device 50, as shown in FIG. The air purification system 55 has a function of evaluating aerosol infection risk using the evaluation device 50 and a function of purifying the air in the space using the air purification device 95. Further, the air cleaning system 55 has a function of maintaining a state in which there is no risk of aerosol infection in the space based on the evaluation result of the risk of aerosol infection by the evaluation device 50.

### <Air Purification Device>

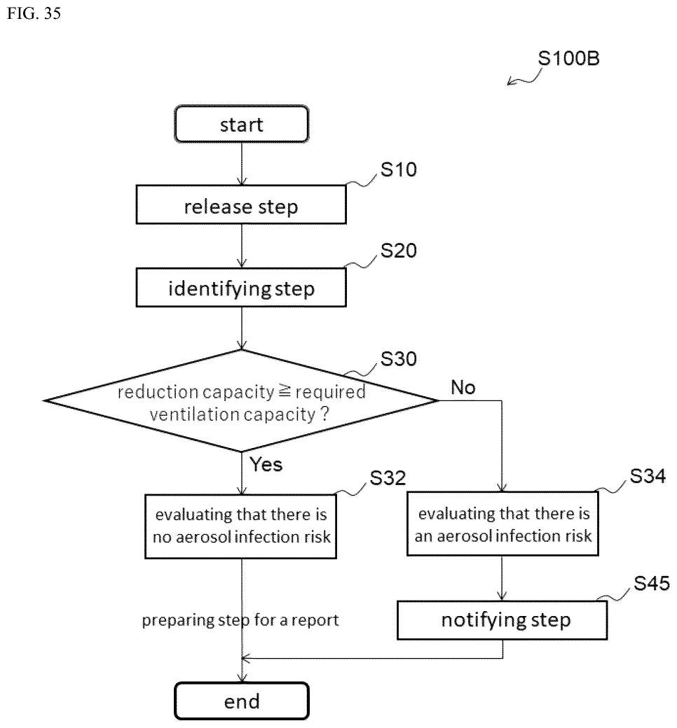

FIG. 35 is a flowchart of the aerosol infection risk evaluation method S100B performed using the air cleaning system 55.

As shown in FIG. 34, the air purifying device 95 includes a fan 95A (an example of an air blowing source), a filter 95B (an example of a trap), and a housing 95C. An intake port 95D and an exhaust port 95E are formed in the housing 95C.

The fan 95A has a function of sucking air into the intake port 95D and exhausting air from the exhaust port 95E. In other words, the fan 95A has a function of sucking air in the space into the casing 95C through the intake port 95D and exhausting it into the space through the exhaust port 95E. Furthermore, since the air purifying device 95 is communicably connected to the evaluation device 50, when the control device 80 makes a negative judgment in the judgment step S30 and proceeds to the negative evaluation step S34, the fan 95A Alternatively, it has a function of increasing the number of rotations and blowing more air under the control of a control device (not shown) of the air purifying device 95. Further, when the control device 80 makes an affirmative judgment in the judgment step S30 and proceeds to the affirmative evaluation step S32, the fan 95A is controlled by the calculation unit 82 or the control device (not shown) of the air purifying device 95, and the rotation speed is It has the function of blowing less air. In this way, by controlling the rotation speed of the fan 95A to increase or decrease, the air cleaning system 55 of this embodiment can attempt to maintain a state in which there is no risk of aerosol infection within the space.

### <Notification Device>

In the notification device AD, when the control device 80 (calculation unit 82) evaluates that there is a risk of aerosol infection in the space, that is, the control device 80 makes a negative judgment in the judgment step S30 and proceeds to the negative evaluation step S34. If so, it has a function of notifying the user of this in the reporting step S45. The notification device AD may be, for example, a light emitting device, a speaker, a monitor, etc., or may be a transmitting device or other device that transmits information to an information terminal used by a user via a network or the like. The notification device AD only needs to be able to notify the user that there is a risk of aerosol infection within the space.

### <Supplement>

Here, the timing at which the air purification system 55 of this embodiment performs the aerosol infection risk evaluation method S100B (see FIG. 35) is determined, for example, by providing a timer (not shown) in the control device 80 and periodically performing the aerosol infection risk evaluation method S100B (see FIG. 35). You can do it like this. Alternatively, the user may send an instruction via a user interface (not shown).

Additionally, the air purifying device 95 may have one or both of a humidification function and a temperature adjustment function. When the air purifying device 95 is provided with a humidifying function, the evaluation may be performed by mixing a plurality of particles into the steam released into the space together with the humidified air.

[Effects of the Air Cleaning System of this Embodiment]

Next, the effects of the air cleaning system 55 of this embodiment will be described.

The air cleaning system 55 of this embodiment can automatically reduce the risk of aerosol infection when it is evaluated that there is a risk of aerosol infection within the space.

Additionally, when the air cleaning system 55 of this embodiment evaluates that there is a risk of aerosol infection within the space, it can notify the user to that effect.

Furthermore, the air purification system 55 of the present embodiment can exhibit an air purification function that reduces the risk of aerosol infection, as well as one or both of a humidification function and a temperature adjustment function. When the air purifying device 95 is provided with a humidifying function, the air purifying device 95 can be provided with the function of the emission source 60. That is, this is effective in that the emission source 60 is not required.

### «8. Modification Example»

As mentioned above, although the present invention has been described using a plurality of embodiments and examples, the present invention is not limited to these embodiments. For example, the technical scope of the present invention also includes the following forms.

For example, in the judgment step S30, the evaluation device 50 described above judges whether the reduction capacity is greater than or equal to the required ventilation capacity (see FIGS. 30 and 31). Any one of the infection risk evaluation methods to the fourth infection risk evaluation method may be used. Alternatively, two or more of these evaluations may be performed, and if one of the evaluation results is positive, it may be determined that there is no risk of infection. Alternatively, for example, two or more of these evaluations may be performed, and if all the evaluation results are positive, it may be determined that there is no risk of infection. In this case, the evaluation criteria can be made stricter. For example, if the evaluation criteria for new viruses that will occur in the future are not clear, performing the latter evaluation will make it easier to ensure safety risks.

Further, for example, the air purifying system 55 described above adjusts the amount of air blown by the fan 95A (see FIGS. 33 and 34), but the air purifying device 95 may be configured as follows. Specifically, the filter 95B is, for example, a plasma cluster type dust collection mechanism (not shown, an example of a trapping part), and when the control device 80 evaluates that there is a risk of aerosol infection in the space, the control device 80 Alternatively, the air purifier may control the plasma cluster type dust collection mechanism to cause the plasma cluster type dust collection mechanism to capture many plural particles.

Furthermore, for example, in the description of this embodiment, the evaluation target is aerosol infection risk, but this embodiment is also applicable to the evaluation of risks other than aerosol infection risk. For example, the present invention may also be used as a dust damage risk evaluation method, evaluation device, and evaluation program for dust damage risk.

### «Supplementary Information Regarding 9.1-3 and 5-8»

Hereinafter, supplementary explanations will be given regarding the plurality of embodiments and modifications described above.

<Quantity of Multiple Liquid Particles Released into Space in Release Step S10>

It is preferable that the quantity is greater than or equal to the saturated steam amount in the space. The reason for this is that if the number of liquid particles (microscopic particles of liquid) released into the space is less than the saturated vapor amount in the space, because of evaporation in the space, multiple particles will be released immediately after release in the release step S10. This is because there is a possibility that a large difference may occur between the number of liquid particles released and the amount of a plurality of liquid particles when measured in the specifying step S20 (see FIG. 35, etc.) for identifying the reduction ability.

Further, the control device 80 preferably operates under the following conditions to measure the quantity of liquid particles over time (for example, at regular time intervals). Specifically, the conditions are (1) that the measuring device 50 records in advance the measurable upper limit of the particle counter 80 in the storage unit 80, and (2) that the concentration of liquid particles in the space to be measured is The calculation unit 82 determines the quantity of liquid particles to be released into the emission source 60 to a predetermined emission amount according to the volume of the space so as not to exceed the concentration corresponding to the measurable upper limit, and (3) After determining the predetermined release amount, the calculation unit 82 sends a release operation command to the release source 60.

<Components of the Plurality of Liquid Particles Released into the Space in the Release Step S10>

In the above embodiment, it was explained that “the components of each liquid particle may include, for example, ethylene glycol and water.” However, the component contained in each liquid particle may be a polyhydric alcohol other than ethylene glycol. (glycerin as an example). The reason for this is as follows.

First, regarding the so-called artificial aerosol generated in the release step S10, it is difficult to generate it unless the liquid has a boiling point higher than water and is relatively easily soluble in water (=difficult to separate). It cannot be used unless it is a “liquid that is safe to handle.” So, for example, dimethyl sulfoxide can be explosive, and dimethyl formamide is toxic if inhaled by humans and can cause eye irritation and genetic diseases. Therefore, polyhydric alcohol is preferable as the liquid other than water contained in the artificial aerosol.

In addition, polyhydric alcohols such as glycerin and glycols have chemical properties such as a relatively high boiling point and a small amount of saturated vapor. Therefore, it can be said to be a suitable artificial aerosol.

In addition, measurements for assessing the risk of aerosol infection based on this embodiment, which fills the space where customers are present with artificial aerosol, may not be possible (or difficult to implement) due to reasons such as the fact that the facility is open and there are customers.) may occur.

Therefore, for example, the following modified measurement method can also be performed. Specifically, the measurement method of this modification is as follows: (1) Instead of the plurality of liquid particles emitted from the emission source 60, a plurality of liquid particles made of volatile organic compounds (VOC) are placed inside the measurement target space. (2) Then, the concentration of volatile organic compounds (VOC) vaporized in the space is measured by a gas concentration detection device (not shown) such as a gas sensor (VOC sensor) instead of the particle counter 70. do. Here, the concentration of volatile organic compounds (VOC) measured by the gas concentration detection device substantially corresponds to the number of liquid particles released from the emission source 60 into the space to be measured.

Air purifiers that decompose chemical substances such as volatile organic compounds (VOCs) are on the market, so even if volatile organic compounds (VOCs) are measured by vaporizing them, air purifiers will not be effective in the same way as artificial aerosols. This is because they are removed by the machine.

In the case of this modification, the quantity of volatile organic compounds (VOC) released into the space from the emission source 60 is preferably less than the saturated vapor amount. The reason for this is that if more than the saturated amount of vapor is released, the liquid will adhere to the floors, walls, etc. that partition the space and gradually vaporize, causing the floors, walls, etc. to become secondary particulate emission sources, and the measurement This is because there is a possibility that an error may occur in the results. Furthermore, among volatile organic compounds (VOCs), the method of vaporizing volatile alcohols such as ethanol can be easily released by using a spray, which reduces the risk of aerosol infection not only in a specific place but in any space. It has the advantage that it can be evaluated easily and simply.

From the above viewpoint, it can be said that the main inventions of each aspect described in the above-mentioned “Means for Solving the Problems” are particularly effective in the following modified examples.

(Invention of a modification of the aerosol infection risk evaluation method of the first aspect) In a space having a predetermined volume, (1) the liquid particles are composed of a volatile organic compound, (2) the average particle size is 10 μm or less, and (3) the number of liquid particles released into the space is volatile. a discharge step of discharging a plurality of liquid fine particles in a quantity corresponding to a quantity less than the saturated vapor quantity of the organic compound; After the discharge step, the quantity of a plurality of liquid particles is measured multiple times in the space, and from the relationship between the elapsed time from the end of the discharge step and the number of the plurality of liquid particles measured, deriving a specific elapsed time until the number of liquid particles decreases to a predetermined number, and specifying a reduction ability of a plurality of liquid particles in the space from the specific elapsed time;
an evaluation step of evaluating that there is a risk of aerosol infection in the space if the reduction capacity is less than the standard reduction capacity required for the space; Methods for assessing aerosol infection risk, including:

### (Invention of a Modification of the Aerosol Infection Risk Assessment Program According to One Embodiment)

to the computer,

In a space having a predetermined volume, (1) the liquid particles are composed of a volatile organic compound, (2) the average particle size is 10 μm or less, and (3) the number of liquid particles released into the space is volatile. a release function that releases a plurality of liquid particles in an amount corresponding to an amount less than the saturated vapor amount of the organic compound; After the discharge of the plurality of liquid particles is completed, the quantity of the plurality of liquid particles is measured multiple times in the space, and the number of the plurality of liquid particles is measured as the elapsed time from the end of the discharge of the plurality of liquid particles. Deriving a specific elapsed time until the number of a plurality of liquid particles decreases to a predetermined number from the relationship with the number of liquid particles in the space, and reducing the plurality of liquid particles in the space from the specific elapsed time. a specific function that specifies the ability;

If the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation function that evaluates that there is no risk of aerosol infection in An aerosol infection risk assessment program.

### (Invention of a Modification of the Aerosol Infection Risk Evaluation Device of the First Aspect)

An aerosol infection risk assessment device, including:
in a space having a defined volume, (1) is composed of a volatile organic compound, (2) has an average particle size of 10 μm or less, and (3) has a quantity equivalent to less than the saturated vapor amount of the volatile organic compound in the space. The quantity of the plurality of liquid particles discharged into the space is measured multiple times, and the elapsed time from the end of the discharge of the plurality of liquid particles is calculated from the number of the plurality of liquid particles to be measured. a specifying unit that derives a specific elapsed time until the number of the plurality of liquid particles decreases to a predetermined amount from the relationship, and specifies a reduction ability of the plurality of liquid particles in the space from the specific elapsed time; if the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation department that assesses that there is no risk of aerosol infection at a control unit that controls the identification unit and the evaluation unit based on the aerosol infection risk evaluation program according to claim 8.

Although each of the above modifications relates to the main aspects of the above-described embodiment, it goes without saying that similar modifications can be made to the aspects that refer to the main aspects.

Additionally, aerosol infection is said to be an infection that is transmitted by droplets (spit) released into the air by humans. Furthermore, it is said that aerosol infection is particularly likely to occur at restaurants that serve alcoholic beverages. In other words, the droplets emitted by people at such restaurants can be said to be droplets of liquid containing water and alcohol (part of alcoholic beverages). From this point of view, the inventor of the present application proposes that each liquid particle constituting the artificial aerosol contains water and alcohol, and that the main components of each liquid particle are water and alcohol. It can be said that the reproducibility is high in evaluating the risk of aerosol infection in restaurants.

From the above viewpoint, it can be said that the main inventions of each aspect described in the above-mentioned “Means for Solving the Problems” are particularly effective in the following modified examples.

### (Invention of Modification of the Aerosol Infection Risk Evaluation Method of the First Aspect)

A method for assessing the risk of aerosol infection at restaurants that serve alcoholic beverages, including:
in a space having a predetermined volume, (1) each liquid particle contains water and polyhydric alcohol, (2) the average particle size is 10 μm or less, and (3) the number of liquid particles in the space is a discharging step of discharging a plurality of liquid particles in an amount equivalent to the saturated vapor amount or more of the polyhydric alcohol;
after the discharge step, the quantity of a plurality of liquid particles is measured multiple times in the space, and from the relationship between the elapsed time from the end of the discharge step and the number of the plurality of liquid particles measured, deriving a specific elapsed time until the number of liquid particles decreases to a predetermined number, and specifying a reduction ability of a plurality of liquid particles in the space from the specific elapsed time;
an evaluation step of evaluating that there is a risk of aerosol infection in the space if the reduction capacity is less than the standard reduction capacity required for the space.

### (Invention of a Modification of the Aerosol Infection Risk Assessment Program According to One Embodiment)

to the computer,

In a space having a predetermined volume, (1) each liquid particle contains water and polyhydric alcohol, (2) the average particle size is 10 μm or less, and (3) the number of liquid particles in the space is a release function that releases a plurality of liquid particles in an amount equivalent to the saturated vapor amount of polyhydric alcohol or more;

After the discharge of the plurality of liquid particles is completed, the quantity of the plurality of liquid particles is measured multiple times in the space, and the number of the plurality of liquid particles is measured as the elapsed time from the end of the discharge of the plurality of liquid particles. Deriving a specific elapsed time until the number of a plurality of liquid particles decreases to a predetermined number from the relationship with the number of liquid particles in the space, and reducing the plurality of liquid particles in the space from the specific elapsed time. a specific function that specifies the ability;

If the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation function that evaluates that there is no risk of aerosol infection in An aerosol infection risk assessment program at restaurants that serve alcoholic beverages.

### (Invention of Modification of the Aerosol Infection Risk Evaluation Device of the First Aspect)

An apparatus for evaluating the risk of aerosol infection at restaurants that serve alcoholic beverages; including:
in a space having a predetermined volume, (1) each liquid particle contains water and polyhydric alcohol, (2) the average particle size is 10 μm or less, and (3) the number of liquid particles in the space is The quantity of a plurality of liquid particles released to correspond to a quantity equal to or more than the saturated vapor amount of polyhydric alcohol is measured multiple times, and the elapsed time from the end of the release of the plurality of liquid particles is measured. Deriving a specific elapsed time until the number of the plurality of liquid particles decreases to a predetermined quantity from the relationship with the number of the plurality of liquid particles, and calculating the number of liquid particles in the space from the specific elapsed time;
if the reduction capacity is equal to or less than the reduction standard capacity required for the space, it is evaluated that there is a risk of aerosol infection within the space, and if the reduction capacity is equal to or greater than the reduction standard capacity, the risk of aerosol infection within the space is evaluated. An evaluation department that assesses that there is no risk of aerosol infection at a control unit that controls the identification unit and the evaluation unit based on the aerosol infection risk evaluation program according to claim 8.

Although each of the above modifications relates to the main aspects of the above-described embodiment, it goes without saying that similar modifications can be made to the aspects that refer to the main aspects.

### INDUSTRIAL APPLICABILITY

The infection risk assessment method of the present embodiment can be used to evaluate the infection risk of infectious diseases in spaces such as cafeterias, restaurants, pubs, cafes, clubs, bars, karaoke boxes, concert venues, etc., reduce the infection risk, and dust. It can be used in work spaces where there is a risk of damage.

Currently, facilities like those where clusters have occurred, and related industries, are being uniformly asked to shorten their working hours or close without evaluating each individual facility, even if they have taken sufficient infection control measures. These methods of countermeasures cause reputational damage and economic losses, and are contributing to the deterioration of society. According to the infection risk evaluation method and infection risk reduction method of the present invention, it is possible to easily and objectively evaluate whether a specific facility has taken infection prevention measures. It will also be possible to make rational decisions and take measures, such as lifting requests for business closures and business closures, which will be extremely beneficial in that it will be possible to balance infection prevention measures with the economy.

The above-mentioned required ventilation capacity and modified required ventilation capacity are standards for determining whether there is an infection risk based on the reduction capacity identified in a specific process. Therefore, it should be noted that in the claims of the present application, the necessary ventilation capacity and the corrected necessary ventilation capacity are specified by the comprehensive name “reduced standard capacity.”

### EXPLANATION OF SYMBOLS

1 Laboratory
2 Smoke machine
4 Multiple liquid particles
6 Particulate measuring device
8 Ventilation fan
10 Air conditioner
12 window
14 Window
16 door
18 Installation stand
50 Evaluation device
55 Air purification system
60 Source of emission
70 Particle counter
80 Control device
82 Arithmetic unit
84 Input section
86 Memory section
88 Output section
90 Report Creation Department
95 Air purification device
95A Fan
95B Filter
95C Housing
95D Intake port
95E Exhaust port
101 Hall
102 Table
104 Chair
106 Particulate meter
108 Ventilation fan
109 Ventilation fan
110 Air purifier
112 Total heat exchanger
114 Air conditioner
116 Entrance
201 Semi-private room
202 Table
204 Chair
206 Particle counter
216 Sliding door
216A crosspiece
220 Wall
222 Wall
226 Wall
228 Wall
224 Window
220A Transom
226A Transom
228A Transom
301 Semi-private room
302 Table
304 Chair
306 Particulate measuring instrument
314 Air conditioner
316 Sliding door
322 Wall
322A Transom
408 Ventilation fan
410 Air purifier
412 Total heat exchanger
414 Air conditioner
416 Sliding door
424 Window
S100 Evaluation method
S100A Evaluation method
S100B Evaluation method
S10 Release process
S20 Specific process
S30 Judgment process
S30A Judgment process
S32 Positive evaluation process
S34 Negative evaluation process
S40 Report creation process
S45 Reporting process
AD Notification device
P Evaluation program

1.-20. (canceled)

21. A method for evaluating an aerosol infection risk, the method comprising:
a release step of releasing a plurality of fine particles having an average particle size of 10 μm or less into a space having a predetermined volume so that the amount in the space is at least 50 mg/m³;
an identifying step includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time; and,
an evaluation step of evaluating that there is an aerosol infection risk in the space when the reduction capacity is less than the standard reduction capacity required for the space.

22. The method for evaluating the aerosol infection risk according to claim 21, wherein
in the evaluation step, evaluating that there is no aerosol infection risk in the space when the reduction capacity is equal to or higher than the standard reduction capacity.

23. The method for evaluating the aerosol infection risk according to claim 21, wherein
the standard reduction capacity is a required ventilation capacity required for the volume of the space or a modified required ventilation capacity obtained by modifying the required ventilation capacity based on the usage mode of at least one or more users who use the space.

24. The method for evaluating the aerosol infection risk according to claim 23, further comprising
a preparing step of preparing a report including the reduction capability identified in the identifying step.

25. The method for evaluating the aerosol infection risk according to claim 24, wherein
the preparing step prepares the report further including information on the two-dimensional spectrum of the relationship.

26. A computer readable storage medium having an evaluating program for evaluating an aerosol infection risk, the evaluating program causing a computer to perform;
a release function of releasing a plurality of fine particles having an average particle size of 10 μm or less into a space having a predetermined volume so that the amount in the space is at least 50 mg/m³;
an identifying function includes measuring the number of a plurality of fine particles in the space a plurality of times after the releasing step, calculating a specific elapsed time until the number of a plurality of fine particles decreases to a predetermined number based on a relationship between the elapsed time from the end of the releasing step and the number of fine particles measured at each elapsed time, and identifying a reduction capacity to reduce a plurality of fine particles in the space from the specific elapsed time; and,
an evaluating function of evaluating that there is the aerosol infection risk in the space when the reduction capacity is less than the standard reduction capacity required for the space and evaluating that there is no aerosol infection risk in the space when the reduction capacity is equal to or higher than the standard reduction capacity.

27. An evaluating apparatus for evaluating an aerosol infection risk, the apparatus comprising;
an identifying device measuring a plurality of times the number of a plurality of fine particles having an average particle size of 10 μm or less released in a space having a predetermined volume so that the amount in the space is at least 50 mg/m³ or more, calculating a specific elapsed time until the number of a plurality of fine particles decreases to a predetermined number based on a relationship between the elapsed time from the end of the releasing step and the number of a plurality of fine particles measured at each elapsed time, and identifying a reduction capacity to reduce a plurality of fine particles in the space from the specific elapsed time;
an evaluating device evaluating that there is an aerosol infection risk in the space when the reduction capacity is less than the standard reduction capacity required for the space and evaluating that there is no aerosol infection risk in the space when the reduction capacity is equal to or higher than the standard reduction capacity; and,
a controller controlling the identifying device and the evaluating device based on the evaluating program for evaluating an aerosol infection risk.
